SEQUENCE LISTING (109 sequences)

SEQ ID NO: 1 - protein, 292 aa, Bacillus pumilus
  REGION 1..292: note = B.pumilus SAFR-032 PrsA. Database Accession CP000813
MKKMALAAVTAVSVLTLGACSSGDKDVIATTKSGDVTKEELYTTLKKQAGGDALNLLVQQKVLADKYKVSDKEIDKKMEEYKKTLGEDRLKQLQDEFGKDYIKDQVKYELLTQKAAKANIKITDKEVKAYYDDLKGKIRASHILVADKKTADEVEKKLDKGEKWDAVVSEYSTDTASAAQGGDVGWFAKEGQMDENFSKAAFKLKVNEISKPVKSQFGYHIIKKTEERGKYDDMKADLKKELLDQKQADTNEIQTILNKLVKDADVKVKDKELENTFKEKSQQPAQQQQQNQ

SEQ ID NO: 2 - DNA, 876 bp, Bacillus pumilus
  source 1..876: mol_type = genomic DNA
  misc_feature 1..876: note = B.pumilus SAFR-032 prsA gene (CDS). Database Accession CP000813
atgaaaaaaatggctttagcagctgtaactgcagttagtgttttaactcttggcgcatgcagtagcggtgataaagatgtcatcgcaacaacaaaatcaggagatgtcacaaaagaagagctttatacaactcttaagaaacaagcaggtggagatgctttaaatctacttgttcaacaaaaggttcttgctgacaaatacaaagtatctgataaagaaattgataagaaaatggaagaatataaaaagacacttggtgaagatcgtttaaaacaacttcaagatgaatttggaaaagattacatcaaagatcaagtgaaatatgagctgctgactcaaaaggctgccaaagctaacatcaaaatcactgataaagaagtaaaagcgtattacgatgacctaaaagggaaaatccgcgcaagccacattcttgtagctgataagaaaacagctgacgaagtggaaaagaaattagataaaggtgaaaaatgggatgctgtagtgtctgaatattcaactgatactgcttcagctgcacaaggcggagatgtaggctggttcgcaaaagaaggtcaaatggacgagaacttcagtaaagctgcattcaaactaaaagtgaatgaaatcagcaaaccagtgaaatcacaattcggctaccatatcattaaaaagaccgaagaacgcggtaaatatgatgacatgaaagcagacttgaaaaaagaactacttgatcaaaaacaagcagacacaaatgaaatccaaaccatcttaaacaaattggtgaaagatgcagatgtgaaagtaaaagataaagagcttgaaaacacgtttaaagaaaaatctcaacaacctgcacaacaacaacaacaaaaccaa

SEQ ID NO: 3 - DNA, 227 bp, Bacillus licheniformis
  source 1..227: mol_type = genomic DNA
  misc_feature 1..227: note = Promoter aprE gene
ctcgggacctctttccctcggaggctgaagcggtctattcatactttcgaactgaacatttttctaaaacagttattaataaccaaaaaattttaaattggtcctccaaaaaaataggcctaccatataattcattttttttctataataaattaacagaataattggaatagattatattatccttctatttaaattattctgaataaagaggaggagagtgagat

SEQ ID NO: 4 - DNA, 249 bp, synthetic construct
  source 1..249: mol_type = other DNA
  misc_feature 1..249: note = functional fragment: gene synthesis construct comprising aprE gene promoter with flanking BpiI sites
gaagaccttcgactcgggacctctttccctcggaggctgaagcggtctattcatactttcgaactgaacatttttctaaaacagttattaataaccaaaaaattttaaattggtcctccaaaaaaataggcctaccatataattcattttttttctataataaattaacagaataattggaatagattatattatccttctatttaaattattctgaataaagaggaggagagtgagatataagtcttc

SEQ ID NO: 5 - DNA, 913 bp, synthetic construct
  source 1..913: mol_type = other DNA
  misc_feature 1..913: note = functional fragment: gene synthesis construct comprising B. pumilus prsA gene with flanking BpiI sites
ggtaccgaagactcatatgaaaaaaatggctttagcagctgtaactgcagttagtgttttaactcttggcgcatgcagtagcggtgataaagatgtcatcgcaacaacaaaatcaggagatgtcacaaaagaagagctttatacaactcttaagaaacaagcaggtggagatgctttaaatctacttgttcaacaaaaggttcttgctgacaaatacaaagtatctgataaagaaattgataagaaaatggaagaatataaaaagacacttggtgaagatcgtttaaaacaacttcaagatgaatttggaaaagattacatcaaagatcaagtgaaatatgagctgctgactcaaaaggctgccaaagctaacatcaaaatcactgataaagaagtaaaagcgtattacgatgacctaaaagggaaaatccgcgcaagccacattcttgtagctgataagaaaacagctgacgaagtggaaaagaaattagataaaggtgaaaaatgggatgctgtagtgtctgaatattcaactgatactgcttcagctgcacaaggcggagatgtaggctggttcgcaaaagaaggtcaaatggacgagaacttcagtaaagctgcattcaaactaaaagtgaatgaaatcagcaaaccagtgaaatcacaattcggctaccatatcattaaaaagaccgaagaacgcggtaaatatgatgacatgaaagcagacttgaaaaaagaactacttgatcaaaaacaagcagacacaaatgaaatccaaaccatcttaaacaaattggtgaaagatgcagatgtgaaagtaaaagataaagagcttgaaaacacgtttaaagaaaaatctcaacaacctgcacaacaacaacaacaaaaccaataatagcttgtcttcgagctc

SEQ ID NO: 6 - DNA, 865 bp, synthetic construct
  source 1..865: mol_type = other DNA
  misc_feature 1..865: note = functional fragment: T2A lacZ cassette
gcgatgttaaggaccgtctcatctcacctgcaaggtctcatctcttttactccatctggatttgttcagaacgctcggttgccgccgggcgttttttatctaaaactagtgtcgagggtcttcggtaccgcgatttacatatgctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgccgcgtttataatgaagaccctagcaggcatcaaataaaacgaaaggctcagtcgaaagactgggcctttcgttttatctgttgtttgtcggtgaacgctctcctgagtaggacaaatccgccgccctagacagctgtcgctgagacgatcgctgagacctcgcaagttctcgccatcgcaggtgaaatctagatgtattcgc

SEQ ID NO: 7 - DNA, 648 bp, Bacillus licheniformis
  source 1..648: mol_type = genomic DNA
  misc_feature 1..648: note = Chloramphenicol acetylransferase (Cat) gene (CDS)
atgaattttcaaacaatcgagcttgacacttggtatagaaaatcctattttgaccattacatgaaggatgcgaaatgttctttctgcatcacggcaaacgtcaatgtgacaaatttgctcgccttgctcaagaaaaagaagatcaagctgtacccggcttttatttatatcgtatcaagggtcattcattcgcgccctgagtttagaacaacttttgatgacaaaggacggctgggttattgggaacaaatgcatccgtgctatgcgatttttcatcaggacgaccaaacgttttccgccctctggacggaatactcagacgatttttcgcagttttatcatcaatatcttctggacgctgagcgctttggagacaaaaggggcctttgggctaagccggacatcccgcccaatacgttctcagtttcatctattccatgggtgagctttacaaacttcaatttaaaccttgataacagcgaacacttgctgccgattatcacaaacgggaaatacttttcagaaggccgggaaacatttttgcccgtttccctgcaagtacaccatgccgtgtgtgacggctatcatgccggcgccttcatgaacgagttggcacggcttgccgccgattgtaaggagtggcttgtg

SEQ ID NO: 8 - DNA, 524 bp, synthetic construct
  source 1..524: mol_type = other DNA
  misc_feature 1..524: note = homology region: 5-prime region of the CAT gene with flanking BsaI sites
ggtctcagagaacctcatcctgtattgtaaatgaattgtagcgcgtttcggaggccgaagcaaccgatccctccttctttgtattcttgctgtgccgttctccgtctctcacccccggctgtcatttaggttatactgacatatatcggggtgaagatatgaacagcaaatcacgattgacgcctgaaatgtggcgggcaattacggaaaacgattccgcctatgacggagttttttattacgcggtcaaaacgaccggcatattttgccgcccatcctgcaaatcgagagttccgcaaatcgacaatgtgcagatttttttcaatgcaaaagatgctttatcagaagggttccgcccctgcaaacgctgcaatcccgccggagcgctgctgccggatgaagagctggcacagcgggtagtgcaaatcatcgagaaatcttatcgcgatacgctgtctctgcaagctttggctgacaggtgccatatcagcccttttcacctgcagcggacatctcatgagacc

SEQ ID NO: 9 - DNA, 424 bp, synthetic construct
  source 1..424: mol_type = other DNA
  misc_feature 1..424: note = homology region: 3-prime region of the CAT gene with flanking BsaI sites
ggtctcaacccaatcctatgtataccggcttttcactccggcttccggcagggtaaataaaaggattggagtgatcaagcaatgccgcaaaacaataacaatcgatatgaactctttttcaaaaaaagatacaaagttctacatatattaaacgattttctcatcggccttttattccttgtcgggagcttctgctttttctccgaggaactgaagccggcgggactatggatgtttgtgatcggcagctttcagctgttaatcaggccgacgatccgtttggttcatgattttcactacagaaagtttgttgagagccggcgttccaaatgccgggagtaaacacaccacagtaagaccgctaagcagcgcatataaaaaaggccgaatcatatcggcctttcctctgtcatgcgacgagacc

SEQ ID NO: 10 - DNA, 23 bp, synthetic construct
  source 1..23: mol_type = other DNA
  misc_feature 1..23: note = Oligonucleotide: PCR-amplification of genomic region of CAT gene
actttcttgaagattcagaattc

SEQ ID NO: 11 - DNA, 22 bp, synthetic construct
  source 1..22: mol_type = other DNA
  misc_feature 1..22: note = Oligonucleotide: PCR-amplification of genomic region of CAT gene
gatatcggaataggagcaagtg

SEQ ID NO: 12 - protein, 286 aa, Bacillus licheniformis
  REGION 1..286: note = B. licheniformis PrsA. Database Accession CP000002
MKKIAIAAITATSVLALSACSGGDSEVVAETKAGNITKEDLYQTLKDNAGADALNMLVQQKVLDDKYDVSDKEIDKKLNEYKKSMGDQLNQLIDQKGEDFVKEQIKYELLMQKAAKDNIKVTDDDVKEYYDGLKGKIHLSHILVKEKKTAEEVEKKLKKGEKFEDLAKEYSTDGTAEKGGDLGWVGKDDNMDKDFVKAAFALKTGEISGPVKSQFGYHIIKKDEERGKYEDMKKELKKEVQEQKQNDQTELQSVIDKLVKDADLKVKDKELKKQVDQRQAQTSSSS

SEQ ID NO: 13 - DNA, 858 bp, Bacillus licheniformis
  source 1..858: mol_type = genomic DNA
  misc_feature 1..858: note = B. licheniformis prsA gene (CDS). Database Accession CP000002
atgaagaagattgcaattgcggcgattacagcgacaagcgtgctggctctcagcgcatgcagcgggggagattctgaggttgttgcggaaacaaaagctggaaatattacaaaagaagacctttatcaaacattaaaagacaatgccggagcggacgcactgaacatgcttgttcagcaaaaagtactcgatgataaatacgatgtctccgacaaagaaatcgacaaaaagctgaacgagtacaaaaaatcaatgggtgaccagctcaaccagctcattgaccaaaaaggcgaagacttcgtcaaagaacagatcaaatacgaacttctgatgcaaaaagccgcaaaggataacataaaagtaaccgatgatgacgtaaaagaatattatgacggcctgaaaggcaaaatccacttaagccacattcttgtgaaagaaaagaaaacggctgaagaagttgagaaaaagctgaaaaaaggcgaaaaattcgaagaccttgcaaaagagtattcaactgacggtacagccgaaaaaggcggcgacctcggctgggtcggcaaagacgataacatggacaaggatttcgtcaaagcggcatttgctttgaaaaccggcgaaatcagcggacctgtgaaatcccaattcggctatcacatcattaaaaaagacgaagaacgcggcaaatatgaagacatgaaaaaagagcttaaaaaagaagtccaagaacaaaagcaaaatgatcaaactgaactgcaatccgtcattgacaaacttgtcaaagatgctgatttaaaagtaaaagacaaagagttgaaaaaacaagtcgaccagcgtcaagctcagacaagcagcagcagc

SEQ ID NO: 14 - protein, 306 aa, Bacillus lentus
  REGION 1..306: note = B. lentus (NCTC4824) PrsA. Database Accession LS483476.1
MKKWVLSLSLAAGVIGLAGCSGGNGEAVVKTSAGDISKEEFYDALKDKYGEQVLQQLVIEKVLSKEYKVSDKELDEQVEEVKAQVGPQFPMVLAQYGYKDEEEFRESLKLSVLQEKAATKDIKVTEKELKEYYENKQPDIEVSHIVVGPEDEKKIKEAKEKLDKGEDFAKVAKEYSIDATAENGGSLGLISRDQPDLDEDFKEAAFKLKKDEISAPIQTQFGWHIILVTDKKEKEPFDKVKADLEKELKKSKLDADVVQNALKAELEKAKLDVKDKDLKAAFDPILKDSEEPADDTKDDAEKEDKK

SEQ ID NO: 15 - DNA, 921 bp, synthetic construct
  source 1..921: mol_type = other DNA
  misc_feature 1..921: note = Synthetic gene variant of B. lentus (NCTC4824) prsA gene (CDS)
atgaaaaagtgggtattatccctttccttagccgctggagtaattggacttgctggctgtagtggaggtaacggagaagcagttgtaaaaacaagtgctggagacatttcaaaagaagaattctatgatgcactaaaagacaaatatggtgaacaagtcttgcaacagcttgtgatcgagaaagtgctttctaaagagtataaagtttcagacaaagaactagacgagcaagtagaagaagtaaaagcacaggtaggtcctcaattcccaatggtgctcgcgcaatatggttataaagatgaagaagaatttcgtgaatctttaaaacttagcgtgcttcaagaaaaagctgctacaaaagatatcaaagtaacggagaaagaacttaaagaatattacgaaaacaaacagcctgatattgaagtaagtcatattgtagtgggaccagaagatgagaagaaaataaaagaagcgaaagaaaagcttgataaaggtgaggattttgctaaagtagctaaagaatattctattgacgctactgctgaaaatggtggaagtctcggtcttatttcaagagatcaacctgatttagacgaagattttaaagaagctgcctttaaattaaaaaaagacgaaatcagcgctcctattcaaacacaatttggttggcacatcattttagtgacagataaaaaagaaaaagagccttttgataaagtgaaagctgatctggaaaaagaacttaaaaaatctaagcttgatgcagatgtcgttcaaaatgcattaaaagctgaacttgaaaaagcaaaattagatgttaaggacaaagaccttaaagctgcattcgatccaattcttaaagatagtgaagaaccggctgacgacaccaaagacgatgcagaaaaagaagataaaaagtaa

SEQ ID NO: 16 - protein, 281 aa, Geobacillus steaothermophilus
  REGION 1..281: note = G. steaothermophilus (CAMR12739) PrsA. Database Accession A0A063YQP9
MKKWMMAAAVVSLMALSACSNDGSEAIVETKNGNITKDEFYNEMKERVGKSVLRDLIDEKVLSKKYKVTDEEIDREIERIKEAYGTQYDLAVQQNGEKVIREMVKLDLLRTKAAVEDIKVTEKELKEYYDNYKPKIRASHILVKDEKTAKEVKAKLDKGEDFAKLAKEYSQDPGSASNGGDLGWFGPGKMVKEFEEAAYKLKVGEVSDPVKTDYGYHIIKVTDKEKKKSFNEMKDEIAFEVKRNKLDPATMQSKVDKLVKDAGVEIKDKDLQDVIGQQGKQ

SEQ ID NO: 17 - DNA, 843 bp, Geobacillus steaothermophilus
  source 1..843: mol_type = genomic DNA
  misc_feature 1..843: note = G. steaothermophilus (CAMR12739) prsA gene (CDS). Database Accession JHUR00000000.1
atgaaaaaatggatgatggccgctgccgtcgtttcgctcatggctttgtcggcctgcagcaacgacggttcggaagcgattgtggaaacgaaaaacggcaatattacgaaagacgaattttataatgagatgaaagagcgcgtcggcaaatccgtccttcgtgatttgattgacgaaaaagtgttgagcaaaaaatacaaggtaaccgatgaagagatcgaccgcgaaatcgaacgaatcaaggaagcgtatggaacgcaatatgatttggccgtgcagcagaacggagaaaaagtgatccgcgaaatggtcaagctcgatttgctgcggacgaaagcggccgttgaagatataaaagtgacggagaaagagctgaaggagtattatgacaactacaagccgaaaattcgcgccagccatattttggtcaaagatgaaaaaacagcaaaagaagtaaaggcgaagcttgataaaggcgaggatttcgccaagttggccaaagaatactcccaagatccaggttcggcgtccaacggcggcgatttaggctggtttggcccagggaaaatggtcaaggagtttgaagaagccgcctataagctgaaagtcggcgaagtgagcgatccggtgaaaacggattacggctaccatatcattaaagtgacggacaaagaaaagaaaaagtcgttcaacgaaatgaaagacgaaattgcgtttgaagtcaaacggaacaagcttgatcctgcgacgatgcagtcaaaagtggacaaactggtgaaagatgccggtgttgagatcaaagacaaagatttgcaagatgtgatcgggcagcaaggaaaacag

SEQ ID NO: 18 - DNA, 40 bp, synthetic construct
  source 1..40: mol_type = other DNA
  misc_feature 1..40: note = Oligonucleotide: PCR-amplification of pBIL009 backbone
caccacaggtctcggggtataatggtttcttagacgtcgg

SEQ ID NO: 19 - DNA, 40 bp, synthetic construct
  source 1..40: mol_type = other DNA
  misc_feature 1..40: note = Oligonucleotide: PCR-amplification of pBIL009 backbone
caccacaggtctcgctcaggtgttgctagtttgttatcag

SEQ ID NO: 20 - DNA, 38 bp, synthetic construct
  source 1..38: mol_type = other DNA
  misc_feature 1..38: note = Oligonucleotide: PCR-amplification of genomic region 5-prime of aprE gene
caccacaggtctcgtgaggcaaaacgcggatcattgga

SEQ ID NO: 21 - DNA, 37 bp, synthetic construct
  source 1..37: mol_type = other DNA
  misc_feature 1..37: note = Oligonucleotide: PCR-amplification of genomic region 5-prime of aprE gene
caccacaggtctcggagagcgtgtgcttccgtgagaa

SEQ ID NO: 22 - DNA, 43 bp, synthetic construct
  source 1..43: mol_type = other DNA
  misc_feature 1..43: note = Oligonucleotide: PCR-amplification of genomic region 3-prime of aprE gene
caccacaggtctcgtcgctagtaaaaagaagcaggttcctcca

SEQ ID NO: 23 - DNA, 43 bp, synthetic construct
  source 1..43: mol_type = other DNA
  misc_feature 1..43: note = Oligonucleotide: PCR-amplification of genomic region 3-prime of aprE gene
caccacaggtctcgaccccgcttgactataacatttccacaca

SEQ ID NO: 24 - DNA, 1183 bp, synthetic construct
  source 1..1183: mol_type = other DNA
  misc_feature 1..1183: note = functional fragment: type-II clone cassette
ggtctcatctcactccatctggatttgttcagaacgctcggttgccgccgggcgttttttatctaatcgagggtcttcggtaccgcgatttacatatgtttataatgaagaccctagccggcgcgccacaaatagcatatagaaaaagctagtgtttttagcactagctttttcttcattctgatgaaggttgtaataaaaaaagagcctgctcattacactgcgggctctttttcatggtcgtaccgttcgtataatgtatgctatacgaagttataataaacccagtatgtcttttggatgaaggcggctttttgtttgacctttattgaccaaaaatgtatgattaaactacatacttaccttttaaggtgaaggaggagccatatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatagctaacacaaaaaagcttgacgggttcccgtcaagctttttgtgtttcaggtatggcgtaacacccggctcaatcaaggccgggtgttttttagatatataacttcgtataatgtatgctatacgaacggtatcgctgagacc

SEQ ID NO: 25 - DNA, 122 bp, synthetic construct
  source 1..122: mol_type = other DNA
  misc_feature 1..122: note = functional fragment: gene synthesis construct comprising secA gene promoter with flanking BpiI sites
gaagaccttcgaaattgtttggaaatgacaaaaggtatgatatgatattgcatatataaaaattactgtttactcatgcttaaacaaggaaattaaagaggagcgttattatataagtcttc

SEQ ID NO: 26 - DNA, 105 bp, synthetic construct
  source 1..105: mol_type = other DNA
  misc_feature 1..105: note = functional fragment: gene synthesis construct comprising signal peptide of B. subtilis aprE gene with flanking BpiI sites (Bacillus subtilis). Database Accession NC_022898
gaagacctatatgagaagcaaaaaattgtggatcagcttgttgtttgcgttaacgttaatctttacgatggcgttcagcaacatgtctgcgcaggctaagtcttc

SEQ ID NO: 27 - DNA, 105 bp, synthetic construct
  source 1..105: mol_type = other DNA
  misc_feature 1..105: note = functional fragment: gene synthesis construct comprising signal peptide of B. subtilis aprE gene with flanking BpiI sites (Bacillus licheniformis). Database Accession CP000002
gaagacctatatgaaacaacaaaaacggctttacgcccgattgctgacgctgttatttgcgctcatcttcttgctgcctcattctgcagcagcggctaagtcttc

SEQ ID NO: 28 - DNA, 1476 bp, synthetic construct
  source 1..1476: mol_type = other DNA
  misc_feature 1..1476: note = functional fragment: gene synthesis construct comprising synthetic alpha amylase gene with flanking BpiI sites
gaagacttggctcatcacaatggcacaaacggtaccatgatgcaatattttgagtggtacctcccaaatgacggaaatcattggaacagattgcgctctgatgcaagcaaccttaaagataagggcataacagctgtttggattccgccagcatggaaaggcgccagtcaaaacgacgtcggatatggagcgtacgatctgtatgatttaggcgagtttaatcagaaaggcacagtaagaacgaaatatggaaccaggaatcaacttcaggcggctgtgacggccctcaagtctaatggcattcaggtgtatggcgacgttgtgatgaaccacaaaggaggagctgatgcgacagaatgggttagagcagtcgaagtaaatcctagcaatcgaaaccaggaggtgagcggtgattacacaatcgaggcatggaccaaattcgactttcctggacgtggcaacacacatagtaattttaagtggagatggtatcacttcgatggcgtagactgggatcaatctagacagttacagaaccggatctacaaatttcgcggaaaaggctgggattgggaagttgatacggagaacggcaattatgattacttgatgtacgcagatatcgacatggatcatccggaagtggtcaatgagttacgtaattggggcgtctggtacactaacacactgggccttgacggattccgcattgatgcggttaaacatatcaaatattcgtttacgcgcgactggttaactcatgtccggaatacaacaggcaagaatatgtttgctgttgcagaattttggaaaaacgatatcggcgcgattgaaaattatctttcaaagactaactggaatcattccgtattcgacgtgcctttgcactacaacctgtataatgcgtcgcgctccggcggtaactatgacatgcgacaaatcttcaacggcacagtcgttcagcgtcaccccacacacgctgttacatttgtagacaaccatgacagccaaccggaggaagctcttgaaagcttcgtagaagaatggtttaagcctctggcctatgcgctgacgcttacgcgggatcaaggatacccgagcgtgttttatggagattactatggcattccgacgcatggcgtgccagccatgaaatcaaaaattgatccgatactcgaagccagacaaaaatatgcatatggtactcaacgggattatttggaccatccggatgtcattggttggacccgtgaaggagatggagtacatgctgactctggcttagccacactgatgtccgatggtcctggaggctcaaaatggatggaagtgggtaagaacaatgcaggtgaagtttggtatgatattaccggcaatcagacgaatacggttactattaataaagatggttggggacaatttcatgtctcaggcggctctgtctcaatctatgttcagcagtagtagcttgtcttc

SEQ ID NO: 29 - protein, 483 aa, synthetic construct
  REGION 1..483: note = Artificial Sequence
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYGTQRDYLDHPDVIGWTREGDGVHADSGLATLMSDGPGGSKWMEVGKNNAGEVWYDITGNQTNTVTINKDGWGQFHVSGGSVSIYVQQ

SEQ ID NO: 30 - protein, 485 aa, synthetic construct
  REGION 1..485: note = Artificial Sequence
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 31 - skipped

SEQ ID NO: 32 - protein, 483 aa, synthetic construct
  REGION 1..483: note = Artificial Sequence
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 33 - skipped

SEQ ID NO: 34 - DNA, 1473 bp, synthetic construct
  source 1..1473: mol_type = other DNA
  misc_feature 1..1473: note = Gene synthesis construct comprising mature alpha amylase gene amyL of B. licheniformis with flanking BpiI sites
gaagacttggctgcaaatcttaaagggacgctgatgcagtattttgaatggtacatgcccaatgacggccaacattggaagcgcttgcaaaacgactcggcatatttggctgaacacggtattactgccgtctggattcccccggcatataagggaacgagccaagcggatgtgggctacggtgcttacgacctttatgatttaggggagtttcatcaaaaagggacggttcggacaaagtacggcacaaaaggagagctgcaatctgcgatcaaaagtcttcattcccgcgacattaacgtttacggggatgtggtcatcaaccacaaaggcggcgctgatgcgaccgaagatgtaaccgcggttgaagtcgatcccgctgaccgcaaccgcgtaatttcaggagaacaccgaattaaagcctggacacattttcattttccggggcgcggcagcacatacagcgattttaaatggcattggtaccattttgacggaaccgattgggacgagtcccgaaagctgaaccgcatctataagtttcaaggaaaggcttgggattgggaagtttccaatgaaaacggcaactatgattatttgatgtatgccgacatcgattatgaccatcctgatgtcgcagcagaaattaagagatggggcacttggtatgccaatgaactgcaattggacggtttccgtcttgatgctgtcaaacacattaaattttcttttttgcgggattgggttaatcatgtcagggaaaaaacggggaaggaaatgtttacggtagctgaatattggcagaatgacttgggcgcgctggaaaactatttgaacaaaacaaattttaatcattcagtgtttgacgtgccgcttcattatcagttccatgctgcatcgacacagggaggcggctatgatatgaggaaattgctgaacggtacggtcgtttccaagcatccgttgaaagcggttacatttgtcgataaccatgatacacagccggggcaatcgcttgagtcgactgtccaaacatggtttaagccgcttgcttacgcttttattctcacaagggaatctggataccctcaggttttctacggggatatgtacgggacgaaaggagactcccagcgcgaaattcctgccttgaaacacaaaattgaaccgatcttaaaagcgagaaaacagtatgcgtacggagcacagcatgattatttcgaccaccatgacattgtcggctggacaagggaaggcgacagctcggttgcaaattcaggtttggcggcattaataacagacggacccggtggggcaaagcgaatgtatgtcggccggcaaaacgccggtgagacatggcatgacattaccggaaaccgttcggagccggttgtcatcaattcggaaggctggggagagtttcacgtaaacggcgggtcggtttcaatttatgttcaaagatagcttgtcttc

SEQ ID NO: 35 - protein, 483 aa, Bacillus licheniformis
  REGION 1..483: note = mature Amylase AmyL Bacillus licheniformis ATCC 14580. Database Accession CP000002
ANLKGTLMQYFEWYMPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEHRIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKAVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 36 - DNA, 1573 bp, synthetic construct
  source 1..1573: mol_type = other DNA
  misc_feature 1..1573: note = Gene synthesis construct comprising amylase A7-7 gene (EP1307547) with flanking BpiI sites
gaagacttatatgagaaaacgtaaaaatggattaatcagtattctattggcatttttgttggtacttacatcaataccttttacttcagcaaacgtagaagcacaccataatggcacaaatggaacaatgatgcaatattttgaatggtatttgccaaatgacggtaatcattggaatagattaagatcagatgcaagtaatcttaaagataaagggattacagcggtttggataccacctgcttggaaaggggcttctcaaaatgatgtagggtatggagcctatgatctgtatgatttaggagaattcaatcaaaaaggaaccgtacgtacaaagtacggaacccgtaatcaattacaagctgcagtaaccgccttaaaaagtaatggtattcaagtatacggagatgtcgtaatgaatcataagggtggagcggatgccactgagtgggttcgagcggttgaagtgaacccaagtaatcgtaatcaagaagtctctggtgattatacgattgaggcttggactaagtttgattttcctggtcgaggtaatacccactctaactttaaatggagatggtatcatttcgatggtgtagattgggatcagtcacgtcaattgcagaatcgaatctataaattcagaggagatggaaaaggttgggactgggaagttgatacagagaacggaaactatgactatctaatgtacgcggatattgatatggatcaccctgaagtagtgaatgaactcagaaactggggtgtatggtataccaatacactggggctagacgggttcagaataggtgcggtaaaacatataaaatatagctttactcgtgattggcttactcacgttagaaatacgacaggtaaaaatatgtttgcagttgcagagttctggaagaatgacataggtgcaattgaaaattacttaagtaaaacaaattggaatcattcagtttttgatgtgcccctgcattataacctttataatgcatcgagaagtggtggcaattatgatatgaggcaaatatttaatggaacagttgttcagagacatcctacacatgctgtaacatttgttgataaccatgattcacagccggaagaagccctagagtcatttgttgaagagtggttcaaaccgttagcgtgtgctctcacactaacacgtgatcaaggatatccttccgttttttatggagattattatgggattccgacgcatggtgtaccagcaatgaaatctaagattgatccgattttagaagcacgtcaaaagtatgcgtacggaaaacaaaatgattatttggatcaccataatatgattggctggacgcgtgaaggtaatacagcacatcccaactcaggactagcaactattatgtcggatggcccaggaggaaataaatggatgtatgttgggcgtaataaggctggacaagtttggagagatattacaggaaatcgctcaggtacggtgacgattaacgcagatgggtggggtaatttttctgtaaatggtgggtctgtatctatatgggtaaataattaatagcttgtcttc

SEQ ID NO: 37 - protein, 516 aa, Bacillus sp.
  REGION 1..516: note = full-length amylase A7-7 gene (EP1307547). Bacillus sp. DSM 12368
MRKRKNGLISILLAFLLVLTSIPFTSANVEAHHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIGAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLACALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 38 - protein, 485 aa, Bacillus sp.
  REGION 1..485: note = mature Amylase A7-7 (EP1307547). Bacillus sp. DSM 12368
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIGAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLACALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 39 - DNA, 1482 bp, synthetic construct
  source 1..1482: mol_type = other DNA
  misc_feature 1..1482: note = Gene synthesis construct comprising gene variant of mature alpha amylase gene WO0060060 (Seq ID 1) with flanking BpiI sites
gaagacttggctcatcataatggcaccaatggaacaatgatgcagtattttgaatggtacctgccgaacgatggaaatcactggaaccgcctgagatccgatgcgtcgaacctgaaagataaaggcatctcagcggtttggattcctcctgcgtggaaaggagcaagccaaaatgatgtcggttatggcgcgtacgatttgtacgatttgggggaatttaaccaaaaaggcacaatccgcacgaagtatggaacgcggaaccagcttcaggccgctgttaatgcgttaaaatcaaacggaatccaggtttatggcgatgttgtcatgaaccataaaggcggagccgatgcaaccgaaatggtcagggccgttgaagtcaatcccaacaatagaaaccaggaggtgtcgggagagtacacaatcgaagcgtggacgaaatttgattttccgggcagaggcaacacgcacagcaattttaaatggcgctggtatcactttgatggagttgattgggatcagagccgcaaacttaataacagaatctataaatttcgcggcgatggcaaaggctgggattgggaggtcgacactgaaaacggcaattacgattacctgatgtatgccgacatcgacatggaccatccggaagttgtcaacgaattgagaaactggggagtgtggtatacgaatacactcgggctggatgggttcaggatagacgcggtaaaacatattaaatatagcttcacgcgcgactggatcaaccatgtccggtccgcgacgggcaaaaacatgtttgcggtggcggaattttggaagaacgatttgggagccatcgaaaactatctgaacaaaacaaactggaatcatagcgtctttgatgtcccgctgcattataacctctataacgcctctaaatctggaggcaactacgacatgcggcaaatttttaacgggacggttgtccagagacatcctatgcacgcagtgacgtttgtcgacaaccatgatagccagccggaagaagcgctggaatcatttgtcgaagaatggttcaaaccgctggcttatgcacttacattaacgcgcgagcagggctatccgtcggtattttatggagattattatggcatcccgacgcatggagtccctgcgatgaaatcaaaaattgatccgatccttgaagccagacaaaaatatgcctatggaaggcagaacgattatcttgaccatcataatattatcggatggacgcgggaaggaaacacggcgcatccgaacagcggcctggcaacaatcatgtcggacggcgcgggcggcaacaaatggatgtttgttggacgtaataaggcgggccaggtgtggacggacatcacaggcaaccgggccggcacagtcacgattaacgccgatggctggggcaacttttccgtcaatggaggatcagtgagcatctgggtcaacaagtaatagcttgtcttc

SEQ ID NO: 40 - protein, 485 aa, Bacillus akibei
  REGION 1..485: note = mature alpha amylase WO0060060 (Seq ID 2)
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 41 - DNA, 1564 bp, synthetic construct
  source 1..1564: mol_type = other DNA
  misc_feature 1..1564: note = Gene synthesis construct comprising synthetic gene of alpha amylase B. cereus (UNIPROT A0A2C1D710_BACCE) with flanking BpiI sites
gaagacttatatgttgaaaaggattatggtagtctgtttattgtttattttactttttcctaatatatatgaggggaataaggcggaagcagcaacgataaacaatggaacattaatgcagtattttgagtggtacgctccgaatgatgggaatcattggaatcgtttgcgttctgatgctgaaaatttagcccataaaggaattacatctgtatggataccacctgcatataaagggacttcacaaaatgatgtaggatatggagcgtatgatttatatgatttgggggaattcaatcaaaaaggaacggtgcggacgaaatatgggacaaaagcacagttgaaatctgcaattgaagctttacataagcaaagcatagatgtgtatggtgatgtagttatgaaccataaaggtggggctgattatactgaaactgtaacagctgtagaggtagaccgaaacaatcgaaatattgaagtatcaggtgattatgaaattaatgcatggacggggtttaactttccagggcgcggagattcttattctaatttcaaatggaaatggtatcattttgacggaacagattgggacgaaggaagaaaattaaatcgcatttataaatttagaggcataggtaaagcgtgggacagggaagtgtccagtgaaaatgggaactatgattatttgatgtatgcagatcttgattttgatcatcctgatgttgcgagagagatgaaaaattggggaatgtggtatgcgaatgaattaaatttagatggctttcgtttagatgctgttaaacatattgatcatgaatatcttcgtgattgggtaaatcatgttagaaagcaaacggggaaggaaatgtttacagtagctgaatattggcaaaatgatattcaggttttaaacaattatttggcgaaggtaaattataatcaatctgtgttcgatgcaccgcttcattacaattttcattatgcttcaaaaggaaatgggaattatgatatgagaaatattttaaatggaacagtaatgcaaaatcacccagcactcgcagttactctcgttgaaaatcatgattcacagcctggccagtcattggaatctgtagtgaatccgtggtttaaaccgttggcatatgcatttattttaacacgtgcagaggggtatccttctgttttctatggtgattactacggtacaaatggaagtagtagctatgaaattccgccgttaaaggataaaattgatccaattttgacggcacgaaaaaactttgcatatggtacgcagcgtgattatttagaccatccagatgtgattggctggacaagagaaggtgatagtgtacatgctaattctggtttagcaacattaatctctgatgggccaggaggaacaaaatggatggatgttggaaagaaaaacgcgggggaagtatggtacgatattacgggtaatcaaacaaatactgtaacaattaataaagatggatgggggcagttccatgtaaatggaggctctgtttcaatatatgttcaacagtaatagcttgtcttc

SEQ ID NO: 42 - protein, 486 aa, Bacillus cereus
  REGION 1..486: note = mature alpha amylase UNIPROT A0A2C1D710_BACCE.
ATINNGTLMQYFEWYAPNDGNHWNRLRSDAENLAHKGITSVWIPPAYKGTSQNDVGYGAYDLYDLGEFNQKGTVRTKYGTKAQLKSAIEALHKQSIDVYGDVVMNHKGGADYTETVTAVEVDRNNRNIEVSGDYEINAWTGFNFPGRGDSYSNFKWKWYHFDGTDWDEGRKLNRIYKFRGIGKAWDREVSSENGNYDYLMYADLDFDHPDVAREMKNWGMWYANELNLDGFRLDAVKHIDHEYLRDWVNHVRKQTGKEMFTVAEYWQNDIQVLNNYLAKVNYNQSVFDAPLHYNFHYASKGNGNYDMRNILNGTVMQNHPALAVTLVENHDSQPGQSLESVVNPWFKPLAYAFILTRAEGYPSVFYGDYYGTNGSSSYEIPPLKDKIDPILTARKNFAYGTQRDYLDHPDVIGWTREGDSVHANSGLATLISDGPGGTKWMDVGKKNAGEVWYDITGNQTNTVTINKDGWGQFHVNGGSVSIYVQQ

SEQ ID NO: 43 - protein, 514 aa, Bacillus cereus
  REGION 1..514: note = full-length alpha amylase UNIPROT A0A2C1D710_BACCE
MAKRVRRITGFVLIFVLTFALLIPAQRSMAADNGTMMQYFEWYLPNDGQHWNKMKNDTAYLSSIGITALWIPPAYKGASQADVGYGAYDLYDLGEFNQKGTVRTKYGTKAELVSAITSLHSKGINVYGDVVMNHKGGADFTENVTAVEVNPNNRNQETSGDYQIQAYTGFNFPGRGNTYSSFKWNWYHFDGTDYDQSRNLNRIYKFRGTGKAWDWEVSTEYGNYDYLLYADVDYDHPDVVNEMKKWGTWYANELKLDGFRIDAAKHIKHSFLGDWVQSVRTSTGKEMFTVAEYWQNNLGSLENYLNKSGNNHSVFDVPLHYNFQAASSQGGYYDMRNLLNGTVTSKQPTRSVTFVDNHDTQPGQALESTVQSWFKPLAYAFILTRESGYPNVFYGDLYGTKGTSGREIPSLKTKIEPLLKARKDFAYGTQRDYIDNQDVIGWTREGNTSKANSGLATLITDGPGGAKRMYVGTQNAGEVWYDLTGNRTDKVTIGSDGWANFNVNGGSVSVYVQQ

SEQ ID NO: 44 - DNA, 1482 bp, synthetic construct
  source 1..1482: mol_type = other DNA
  misc_feature 1..1482: note = Gene synthesis construct comprising mature part of alpha amylase gene Cytophaga sp (Genbank AF067653.1) with flanking BpiI sites
gaagacttggctgctgcaacaaatggaacgatgatgcaatattttgaatggtatgttccaaatgacggccagcaatggaaccgattgcgcaccgatgctccgtatttatcaagcgttggcataacagcggtttggacgccgcctgcatacaagggaacaagtcaagcggatgttggatacggaccgtacgatttgtacgatctaggggaatttaatcaaaaaggcaccgtgcgtacaaagtacggaacgaaaggcgagttaaaatccgctgttaatacgcttcattcaaacggtatacaggtttatggagacgtggttatgaaccacaaagcgggggcagactacacggaaaacgtaacggcggttgaagtaaatccgagcaatcgaaatcaagaaacgtctggagaatataatattcaagcttggacgggttttaacttcccgggacgaggaacgacctatagtaattttaaatggcagtggtttcattttgacggaacggactgggatcaatcgagaagcttgagccgcatctttaagtttaggggaaccggcaaagcatgggattgggaggtatcatcagagaacggaaattatgattatttaatgtatgcagatattgattacgatcatcccgacgttgtaaacgagatgaaaaaatggggcgtatggtatgcaaatgaagtgggattagatggataccgcctagatgcggtgaaacatattaagttttcgttcttaaaagactgggtggataatgcgcgtgcagccactggaaaagaaatgtttacggtaggagagtactggcagaatgatctaggtgcactcaacaactatctagcaaaggttaattacaatcagtcgctatttgatgcaccgcttcactacaatttttatgcggcatcaacgggtggcgggtattatgacatgcgaaatatcttaaacaatacgctagtcgcttctaacccaacaaaagcggtgacgctcgttgagaatcatgacacacagccgggccaatcactagaatcaacggttcagccttggtttaagccgttagcctatgcgtttattttaacgagatctggcgggtatccatccgtattctatggcgatatgtacggaacaaaaggtacgacaacacgcgaaattccagcattgaagtcgaaaattgaaccattgctgaaagcgagaaaagactatgcttatggtacgcagcgtgattacatcgataatccagatgtgattggatggactcgagaaggagatagtacaaaagcaaaatctggacttgcgacggtcattaccgatggtcctggcggatcgaagcgaatgtatgtgggaacgtctaatgcgggagaaatctggtatgatctcacaggaaatcgaacagataaaattacgattggatcagatggttatgcaacgtttcctgtaaacggtggttctgtttctgtatgggttcagcagtaatagcttgtcttc

SEQ ID NO: 45 - protein, 486 aa, Cytophaga sp; identical to SEQ ID NO: 42
  REGION 1..486: note = mature alpha amylase Cytophaga sp (Genbank AAF00567.1)

SEQ ID NO: 46 - protein, 519 aa, Cytophaga sp
  REGION 1..519: note = full-length alpha amylase Cytophaga sp (Genbank AAF00567.1)
MKKKWKKRSRFAAITMFSTMLLVPSLAQPKEAKAAATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRNQETSGEYNIQAWTGFNFPGRGTTYSNFKWQWFHFDGTDWDQSRSLSRIFKFRGTGKAWDWEVSSENGNYDYLMYADIDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGYYDMRNILNNTLVASNPTKAVTLVENHDTQPGQSLESTVQPWFKPLAYAFILTRSGGYPSVFYGDMYGTKGTTTREIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 47 - DNA, 1473 bp, synthetic construct
  source 1..1473: mol_type = other DNA
  misc_feature 1..1473: note = Gene synthesis construct comprising gene variant of mature part of alpha amylase from Bacillus sp. TS-23. (uniprot ID Q59222) with flanking BpiI sites
gaagacttggctaacacagcgccgatcaatgaaacaatgatgcagtactttgagtgggacttgccaaatgatggcacgctatggaccaaggtaaaaaatgaagcggcaaaccttagctctcttggcatcacagcgctttggttacctccggcttataaaggaacatcacagtctgacgtcggatatggcgtgtacgatctctacgacctgggtgaatttaatcaaaaaggcacaatccgtacgaagtatggtacgaaaactcaatatattcaggcgattcaagcggctaaagcggcaggcatgcaagtctacgcggatgtcgtatttaatcataaagcaggagcagatggaacagaatttgtagatgctgttgaagtcgatccttccaaccgcaatcaggaaaccagtggtacatatcaaattcaggcttggaccaagttcgattttccgggaagaggcaacacttacagttccttcaaatggcgttggtaccattttgatggaacagattgggacgaaagccggaaacttaaccgaatatataagtttcgtggcaaggcttgggactgggaagttgataccgagaacggcaactacgactatttaatgtttgcggacctagatatggatcatccggaggtggttactgaactgaaaaactggggcacgtggtatgtaaatacaactaacattgatggcttccgattagacgctgtgaaacatattaagtacagctttttccctgattggcttacgtatgttcggaatcaaacgggcaaaaatctgtttgcagtgggagagttctggtcgtacgatgtaaacaagctccataattatattacaaaaacaaacggatccatgagcttatttgacgccccgcttcacaacaatttttataccgcttcaaaaagctcaggatattttgatatgagatacttactgaataacacattaatgaaggatcaaccaagcctggcagtcacacttgtggacaatcacgatacccaacctggtcagtccttgcagtcatgggtggaaccgtggtttaaaccactggcatatgcattcattctgacaagacaggagggctatccttgcgttttctatggcgattattacggcattcccaaatataatattccgggccttaaatctaaaattgatccgttgttaattgccaggcgcgattatgcctatggcacgcagagagattacattgatcaccaagacatcattggctggacgcgcgaaggaatagacactaaaccgaattctggactcgccgctttgattacggacggcccaggcggatcaaagtggatgtatgtgggaaaaaagcatgccggtaaagtcttttatgatctcacaggcaatcggagcgatactgtgacaataaatgccgacggttggggagaatttaaagtgaacggaggttccgtttcaatctgggttgcgaaataatagcttgtcttc

SEQ ID NO: 48 - protein, 482 aa, synthetic construct
  REGION 1..482: note = Protein variant of mature part of alpha amylase from Bacillus sp. TS-23. (uniprot ID Q59222)
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 49 - DNA, 1561 bp, synthetic construct
  source 1..1561: mol_type = other DNA
  misc_feature 1..1561: note = Gene synthesis construct comprising gene variant of the alpha amylase from Bacillus coahuilensis (WP_059351662.1) with flanking BpiI sites
gaagacttatatgaaattgaaaaagcagttgttggcaattggcttaattgtgttgttaagttttagtcctcttgggacaagtttagctagtgcaaatacaaatggcacgatgatgcaatatttcgaatggtatttgccaaatgatggacaacattggaacagattgaataatgatgccgctcatctacaagatttaggagtaacagctgtatggataccaccggcatataaaggaacatctcaaaatgatgtaggctacggagcctatgatttatatgaccttggagaattcaatcaaaaaggaaccacacgcacaaaatacggaaccaaagctcaattaaaacaggcaatttctagtcttcacaaccgaggtatacaagtatacggggatgtcgtgatgaatcataagggtggagcagattatacgcaaatggtttcagcagttgaggtagatcgttataatcgaaatcaagaagttactggtgaatatcaaatccaggcttggacaggttttaatttccctggtagaaataacacgtattcaccgtttaaatggcaatggtatcattttgacggaacagattgggatcagtcacgtagcttaaatcgaatttacaagtttagaggaacaggaaaagcatgggactgggaagtcgcttctgaatatggtaactatgattatttaatgtatgcagatgttgattttgatcacccagacgttcaacaagagatgaaaaattggggtacatggtatgccaatgaacttggattagatggctttagattagacgcagtgaagcatattaaacattcctatctgcaagattgggtaacggctgtaaggcaaaacactgggaaggaaatgttcacagtagcagaatactggcaaaacgaccttggggctattcaaaactatctaactaaaacaggatatactcactccgcatttgatgtaccactacattacaattttcagcaagcatcgaatagtagcggtagttatgatatgagaaacctgttaaacggtacattaatggctgctaatcctacgaaagcagttaccattgttgaaaatcatgattctcaacctgggcaagcgttagaatctacagttcaatcgtggtttaagccactggcttatgcctttatattaacaagacaggaaggatatcctgccctattttatggagactattatggtacaaagggaacaacttcttatgaaattcctgctttaggtagtaagcttgatcccctattacaggcacgtaaatcctatgcttatggttctcagcatgattacttaaatcactgggacattatagggtggactcgtgagggagttagctcacgtcagaaatcaggattagctacactgattacagatggacctggtggaaacaagtggatgtatgttgggaaacaaaatgcaggggagacatggaaagacatgacagggaaccgctctgatcaggtagtcattaaccaggatggctgggggaacttttatgttaacggaggctctgtctctgtatatgtacagcaataatagcttgtcttc

SEQ ID NO: 50 - protein, 512 aa, Bacillus coahuilensis
  REGION 1..512: note = Full length alpha amylase from Bacillus coahuilensis. Database Accession WP_059351662.1
MKLKKQLLAIGLIVLLSFSPLGTSLASANTNGTMMQYFEWYLPNDGQHWNRLNNDAAHLQDLGVTAVWIPPAYKGTSQNDVGYGAYDLYDLGEFNQKGTTRTKYGTKAQLKQAISSLHNRGIQVYGDVVMNHKGGADYTQMVSAVEVDRYNRNQEVTGEYQIQAWTGFNFPGRNNTYSPFKWQWYHFDGTDWDQSRSLNRIYKFRGTGKAWDWEVASEYGNYDYLMYADVDFDHPDVQQEMKNWGTWYANELGLDGFRLDAVKHIKHSYLQDWVTAVRQNTGKEMFTVAEYWQNDLGAIQNYLTKTGYTHSAFDVPLHYNFQQASNSSGSYDMRNLLNGTLMAANPTKAVTIVENHDSQPGQALESTVQSWFKPLAYAFILTRQEGYPALFYGDYYGTKGTTSYEIPALGSKLDPLLQARKSYAYGSQHDYLNHWDIIGWTREGVSSRQKSGLATLITDGPGGNKWMYVGKQNAGETWKDMTGNRSDQVVINQDGWGNFYVNGGSVSVYVQQ

SEQ ID NO: 51 - protein, 484 aa, Bacillus coahuilensis
  REGION 1..484: note = Mature alpha amylase from Bacillus coahuilensis. Database Accession WP_059351662.1
NTNGTMMQYFEWYLPNDGQHWNRLNNDAAHLQDLGVTAVWIPPAYKGTSQNDVGYGAYDLYDLGEFNQKGTTRTKYGTKAQLKQAISSLHNRGIQVYGDVVMNHKGGADYTQMVSAVEVDRYNRNQEVTGEYQIQAWTGFNFPGRNNTYSPFKWQWYHFDGTDWDQSRSLNRIYKFRGTGKAWDWEVASEYGNYDYLMYADVDFDHPDVQQEMKNWGTWYANELGLDGFRLDAVKHIKHSYLQDWVTAVRQNTGKEMFTVAEYWQNDLGAIQNYLTKTGYTHSAFDVPLHYNFQQASNSSGSYDMRNLLNGTLMAANPTKAVTIVENHDSQPGQALESTVQSWFKPLAYAFILTRQEGYPALFYGDYYGTKGTTSYEIPALGSKLDPLLQARKSYAYGSQHDYLNHWDIIGWTREGVSSRQKSGLATLITDGPGGNKWMYVGKQNAGETWKDMTGNRSDQVVINQDGWGNFYVNGGSVSVYVQQ

SEQ ID NO: 52 - DNA, 2085 bp, synthetic construct
  source 1..2085: mol_type = other DNA
  misc_feature 1..2085: note = Gene synthesis construct comprising gene variant of mature part of maltogenic alpha amylase gene amyM (Geobacillus stearothermophilus, WO9943794-0002) with flanking BpiI sites
gaagacttggcttcgtcgtctgcatccgttaaaggcgacgtcatctatcagatcattatcgatcggttttatgatggcgacaccaccaataacaaccctgccaaatcttatggcctgtacgatcctacaaaaagcaaatggaaaatgtactggggcggggacttagaaggcgtccgccagaaactcccgtatttgaaacagctgggcgtcacgaccatttggcttagcccggtccttgataaccttgacacgctggcgggcacggataatacgggataccatgggtactggacgagagattttaaacagatcgaggaacattttggaaactggacgacatttgatacgctcgtcaacgatgcgcatcaaaacggcatcaaagtaattgtcgactttgtcccgaaccacagcacgccgtttaaagcaaacgatagcacgttcgcggaaggcggagcgctttataacaacggcacctacatgggaaactattttgatgatgcaacgaaaggctattttcaccataatggagacatctcaaactgggatgatcggtatgaagcgcagtggaaaaactttacagatcccgcaggatttagccttgcggacctgtcacaggaaaatggcacgattgcgcagtatctgaccgatgccgcggtccaactggttgcgcatggagcggatggcttgcggattgatgcagtgaaacattttaactcaggctttagcaaatcattggcagataaactttatcaaaagaaagacatctttcttgtcggcgaatggtatggcgatgatccaggcacggcaaaccatttggagaaagtccgctacgctaataacagcggcgttaatgttcttgattttgacttaaataccgtcattaggaatgtctttggaacatttacacagacgatgtacgatctcaataacatggtgaaccaaacgggaaacgaatataagtataaagagaacctcatcacattcattgacaaccacgatatgtcaagattcctttcagttaacagcaacaaagcgaacttgcatcaggccctggcgtttattctgacaagcagaggaaccccgtcgatctattatggaacagaacagtatatggccggcgggaatgatccgtataacagaggcatgatgccggcgtttgacacaacgacaacggcgtttaaggaagtcagcacgctcgcgggcctccgtcgcaataacgcggcgatccagtatggcacaacaacacagcgctggatcaataatgatgtgtatatttacgaacgcaaatttttcaacgatgttgtccttgttgcgatcaataggaacacgcaatcgtcttattccatctcaggattgcaaaccgccctgccgaacggaagctatgccgattatctgagcggccttctgggcggtaatggcatcagcgtcagcaacggatcagttgcctccttcacactggccccgggagctgtcagcgtatggcagtactcgacaagcgcgtctgcaccgcagatcggctcagtcgctccgaatatgggcatccccggcaacgtggttaccatcgatggcaaaggatttgggacaacgcagggaacggttacatttggtggcgtgacggccacggtcaaaagctggacgtcgaaccgcattgaagtttacgtcccgaatatggccgcgggcctgacagatgtcaaagttacggccggaggcgtctccagcaacctgtattcgtataatatccttagcggtacgcagacgtcagtcgttttcacggtgaaatctgcgccgccgacaaacttaggagataaaatttatttgacgggcaacatcccggaacttggaaattggtctacagatacaagcggggccgtcaacaatgcgcagggccctctgcttgcccctaactatccggattggttttatgtcttttcggtgcctgcggggaaaacgatccaatttaagttctttataaagagagccgacggaacgatccagtgggaaaacggatcaaaccatgttgccacgactcctacgggcgcaacagggaacatcactgtcacgtggcaaaactaatagcttgtcttc

SEQ ID NO: 53 - protein, 686 aa, synthetic construct
  REGION 1..686: note = Mature part of maltogenic alpha amylase SEQ ID 2 of WO9943794 - a variant of amyM (Geobacillus stearothermophilus)
SSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 54 - protein, 719 aa, synthetic construct
  REGION 1..719: note = Full length maltogenic alpha amylase SEQ ID 2 of WO9943794 - a variant of amyM (Geobacillus stearothermophilus)
MKKKTLSLFVGLMLLIGLLFSGSLPYNPNAAEASSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 55 - DNA, 1905 bp, synthetic construct
  source 1..1905: mol_type = other DNA
  misc_feature 1..1905: note = Gene synthesis construct comprising mature part of alpha amylase (WP_014416850.1) with flanking BpiI sites
gaagacttggctgaaactgcacacaaatcgaatgaggtgaccgattcatcggtcaaaaacgggaccatccttcatgcatggaattggtcattcaatacgttaacagaaaatatgaaagagattcgtgatgcgggttatgcagccattcagacgtctccgattaaccaagtaaaggaagggaaccaaggagataaaagcatgtcgaactggtactggctctatcagccgacatcgtaccaaatcggcaaccgttacttaggcactgaacaagaatttaaggacatgtgtgcagccgcggaaaaatatggcgtaaaagtcattgtcgatgcggttgtcaatcataccaccagcgattatggcgcgatttctgacgagattaagcgtattccaaactggacccatggaaacacacaaattaaaaattggtcggaccgatgggacatcactcaaaatgcattgcttgggctgtatgattggaatactcagaatactgaggtgcaagcctacctgaaaggtttcttggaaagagcattgaatgacggagcagacgggttccgctatgatgccgccaagcatatagagcttccggatgatgggaattacggcagccaattttggccgaatatcacaaatacatcggcggagttccaatacggagaaatcctgcaagacagcgcgtccagagatactgcttatgcgaattatatgaatgtgacggcttctaactatgggcattccatcagatccgctttaaagaaccgtaatctgagtgtgtcgaatatctcccattatgcatctgatgtatctgcggacaagttagtcacatgggtggaatcacatgatacgtatgccaatgatgatgaagagtctacatggatgagtgatgacgatattcgtttaggctgggcagtgattggttcgcgctcaggaagcacgcctcttttcttttccaggcctgagggcggaggaaatggtgtaagatttcccggaaaaagtcaaataggagatcgcgggagcgccttatttaaagatcaggcgatcactgcggtcaaccaatttcacaatgaaatggccgggcagcctgaggaactctcaaatccgaatgggaacaatcaaatatttatgaatcagcgcggctcaaaaggcgttgtgctggcaaatgcaggatcatcttctgtcaccatcaatacttcaacgaaattacctgacggcaggtatgataatagggccggcgccggttcatttcaagtagcgaacggcaaactgacaggtacgatcaatgccagatccgcggctgttctttatcctgatgatattggaaatgcgcctcatgtctttcttgagaattaccaaacagaggcagtccattctttcaatgatcagctgacggtcaccctgcgtgcaaatgcgaaaacagcaaaagccgtttaccaaatcaataatgggcaggagacagcatttaaggatggagatcgattaacgatcgggaaagaagatccaatcggcacgacatacaacgtcaagttaaccggaacgaacggcgagggtgcatcgagaacccaagaatacacgtttgtcaaaaaagacccgtctcaaaccaacatcattggctatcaaaatccggatcattggggccaggtaaatgcttatatctataaacatgatggaggtggggccatagaattaaccggatcgtggccggggaaagccatgactaagaatgcagatggaatttacacgctgacgctgcctgcgaatgcggatacggccgacgcaaaagtgatttttaacaatggcagcgcccaagtgcccggacagaaccagcccggctttgattatgtgcagaatggtttgtataacaactctggtttgaatggttatcttacgcattaatagcttgtcttc

SEQ ID NO: 56 - protein, 659 aa, Bacillus sp.
  REGION 1..659: note = Bacillus multi species amylase. Database Accession WP_014416850.1
MFKKRFKTSLLPLFAGFLLLFHLVLSGPAAANAETAHKSNEVTDSSVKNGTILHAWNWSFNTLTENMKEIRDAGYAAIQTSPINQVKEGNQGDKSMSNWYWLYQPTSYQIGNRYLGTEQEFKDMCAAAEKYGVKVIVDAVVNHTTSDYGAISDEIKRIPNWTHGNTQIKNWSDRWDITQNALLGLYDWNTQNTEVQAYLKGFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNQFHNEMAGQPEELSNPNGNNQIFMNQRGSKGVVLANAGSSSVTINTSTKLPDGRYDNRAGAGSFQVANGKLTGTINARSAAVLYPDDIGNAPHVFLENYQTEAVHSFNDQLTVTLRANAKTAKAVYQINNGQETAFKDGDRLTIGKEDPIGTTYNVKLTGTNGEGASRTQEYTFVKKDPSQTNIIGYQNPDHWGQVNAYIYKHDGGGAIELTGSWPGKAMTKNADGIYTLTLPANADTADAKVIFNNGSAQVPGQNQPGFDYVQNGLYNNSGLNGYLTH

SEQ ID NO: 57 - protein, 626 aa, Bacillus sp.
  REGION 1..626: note = mature part of Bacillus multi species amylase. Database Accession WP_014416850.1
ETAHKSNEVTDSSVKNGTILHAWNWSFNTLTENMKEIRDAGYAAIQTSPINQVKEGNQGDKSMSNWYWLYQPTSYQIGNRYLGTEQEFKDMCAAAEKYGVKVIVDAVVNHTTSDYGAISDEIKRIPNWTHGNTQIKNWSDRWDITQNALLGLYDWNTQNTEVQAYLKGFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNQFHNEMAGQPEELSNPNGNNQIFMNQRGSKGVVLANAGSSSVTINTSTKLPDGRYDNRAGAGSFQVANGKLTGTINARSAAVLYPDDIGNAPHVFLENYQTEAVHSFNDQLTVTLRANAKTAKAVYQINNGQETAFKDGDRLTIGKEDPIGTTYNVKLTGTNGEGASRTQEYTFVKKDPSQTNIIGYQNPDHWGQVNAYIYKHDGGGAIELTGSWPGKAMTKNADGIYTLTLPANADTADAKVIFNNGSAQVPGQNQPGFDYVQNGLYNNSGLNGYLTH

SEQ ID NO: 58 - DNA, 1473 bp, synthetic construct
  source 1..1473: mol_type = other DNA
  misc_feature 1..1473: note = Gene synthesis construct comprising gene variant of mature part of alpha amylase (WO9526397 SEQ ID No 5) with flanking BpiI sites
gaagacttggctcatcataatgggacaaatgggacgatgatgcaatactttgaatggcacttgcctaatgatgggaatcactggaatagattaagagatgatgctagtaatctaagaaatagaggtataaccgctatttggattccgcctgcctggaaagggacttcgcaaaatgatgtggggtatggagcctatgatctttatgatttaggggaatttaatcaaaaggggacggttcgtactaagtatgggacacgtagtcaattggagtctgccatccatgctttaaagaataatggcgttcaagtttatggggatgtagtgatgaaccataaaggaggagctgatgctacagaaaacgttcttgctgtcgaggtgaatccaaataaccggaatcaagaaatatctggggactacacaattgaggcttggactaagtttgattttccagggaggggtaatacatactcagactttaaatggcgttggtatcatttcgatggtgtagattgggatcaatcacgacaattccaaaatcgtatctacaaattccgaggtaaggcatgggattgggaagtagattcggaaaatggaaattatgattatttaatgtatgcagatgtagatatggatcatccggaggtagtaaatgagcttagaagatggggagaatggtatacaaatacattaaatcttgatggatttaggatcgatgcggtgaagcatattaaatatagctttacacgtgattggttgacccatgtaagaaacgcaacgggaaaagaaatgtttgctgttgctgaattttggaaaaatgatttaggtgccttggagaactatttaaataaaacaaactggaatcattctgtctttgatgtcccccttcattataatctttataacgcgtcaaatagtggaggcaactatgacatggcaaaacttcttaatggaacggttgttcaaaagcatccaatgcatgccgtaacttttgtggataatcacgattctcaacctggggaatcattagaatcatttgtacaagaatggtttaagccacttgcttatgcgcttattttaacaagagaacaaggctatccctctgtcttttatggtgactactatggaattccaacacatagtgtcccagcaatgaaagccaagattgatccaatcttagaggcgcgtcaaaattttgcatatggaacacaacatgattattttgaccatcataatataatcggatggacacgtgaaggaaataccacgcatcccaattcaggacttgcgactatcatgtcggatgggccagggggagagaaatggatgtacgtagggcaaaataaagcaggtcaagtttggcatgacataactggaaataaaccaggaacagttacgatcaatgcagatggatgggctaatttttcagtaaatggaggatctgtttccatttgggtgaaacgatagcttgtcttc

SEQ ID NO: 59 - protein, 483 aa, synthetic construct
  REGION 1..483: note = Protein variant of mature part of alpha amylase (WO9526397 SEQ ID No 5)
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGKAWDWEVDSEFGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 60 - DNA, 1476 bp, synthetic construct
  source 1..1476: mol_type = other DNA
  misc_feature 1..1476: note = Gene synthesis construct comprising gene variant of mature part of alpha amylase (WO2020188095) with flanking BpiI sites
gaagacttggctcatcacaatggcacgaatggaacaatgatgcaatatttcgaatggtatcttccgaacgacggaaatcattggaatagacttaattctgacgcgagcaatttgaagtctaaaggaattaccgcagtttggatccccccagcatggaaaggcgcatctcaaaatgatgtgggctacggagcatacgatctgtacgatcttggagagttcaaccagaaaggcacggtgcgcactaaatatggcaccaggagccaacttcaggctgcggtaacctcactcaaaaataacggcattcaggtgtatggagatgttgtaatgaatcataaaggtggagcagatgcgacggaaatggtaagagctgtcgaggttaacccgaacaatagaaaccaggaggtgacaggcgaatacactatcgaggcatggacaagatttgactttccgggtcggggtaacacacatagctcatttaaatggcgctggtatcattttgatggcgttgattgggatcagagccgtcgccttaataacagaatctataagttcaggggaaaagcctgggactgggaagtcgacactgagaatggaaactatgattatctcatgtatgccgacatagatatggaccacccggaagtggttaatgaacttcggaattggggcgtttggtatacgaacacgttaggactggatggttttagaatcgatgctgttaaacacatcaaatattcgtttacacgtgattggattaatcatgtccggtcggcaacaggaaagaacatgtttgctgtggccgaattctggaagaacgatctgggcgctattgagaactacttacaaaaaacgaattggaaccattcagtctttgatgtacctctgcattataacttatataatgccagtaaatctggcggaaactatgacatgagaaatatatttaacggaacagtggtccagcgacatccatctcatgcggttacatttgtggataatcatgactctcaacccgaagaggccttagaatcattcgtggaagagtggttcaagccgttggcgtacgcgttgaccctaacacgcgaacagggctaccctagcgttttttatggcgattactatggtatccctactcacggcgtcccagctatgcgctccaagattgacccgatcctcgaagcgcgacaaaaatacgcatatggtccgcaacatgattatctggatcaccctgacgtaattggatggacgcgtgaaggagatagttcacaccctaaatccggcctggcgacattgattacggatggtccgggcggatccaaacgtatgtatgctggcttaaaaaatgccggcgaaacatggtatgacattacaggaaatcggtcggataccgtcaagattggcagcgacggttggggcgaatttcatgtcaatgatggctccgtctcaatttatgtacaaaaataatagcttgtcttc

SEQ ID NO: 61 - protein, 483 aa, synthetic construct
  REGION 1..483: note = Protein variant of mature part of alpha amylase (WO2020188095)
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGPQHDYLDHPDVIGWTREGDSSHPKSGLATLITDGPGGSKRMYAGLKNAGETWYDITGNRSDTVKIGSDGWGEFHVNDGSVSIYVQK

SEQ ID NO: 62 - protein, 486 aa, synthetic construct
  REGION 1..486: note = Artificial Sequence
ATINNGTLMQYFEWYAPNDGNHWKRLHTDAGNLAQKGITSVWIPPAYKGTTQNDVGYGAYDLYDLGEFNQKGTVRTKYGTKAQLKSAIEALHKQNINVYGDVVMNHKGGADYTETVTAVEVDRNNRNIEVSGDYEINAWTGFNFPGRGDTHSNFKWKWYHFDGTDWDEGRKLNRIYKFRGIGKAWDWEVSSENGNYDYLMYADLDFDHPDVANEMKNWGTWYANELNLDGFRLDAVKHIDHEYLRDWVNHVRQQTGKEMFTVAEYWQNDIQTLNNYLAKVNYNQSVFDAPLHYNFHYASKGNGNYDMRNILNGTVMQNHPALAVTLVENHDSQPGQSLESVVSPWFKPLAYAFILTRAEGYPSVFYGDYYGTSGNSSYEIPALKDKIDPILMARKNFAYGTQRDYLDHPDVIGWTREGDGVHADSGLATLISDGPGGSKWMEVGKNNAGEVWYDMTGNQTNTVTINKDGWGQFHVSGGSVSIYVQQ

SEQ ID NO: 63 - protein, 399 aa, synthetic construct
  REGION 1..399: note = Artificial Sequence
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYG

SEQ ID NO: 64 - protein, 86 aa, synthetic construct
  REGION 1..86: note = Artificial Sequence
KQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 65 - protein, 86 aa, synthetic construct
  REGION 1..86: note = Artificial Sequence
TQRDYLDHPDVIGWTREGDGVHADSGLATLISDGPGGSKWMEVGKNNAGEVWYDMTGNQTNTVTINKDGWGQFHVSGGSVSIYVQQ

SEQ ID NO: 66 - protein, 400 aa, synthetic construct
  REGION 1..400: note = Artificial Sequence
ATINNGTLMQYFEWYAPNDGNHWKRLHTDAGNLAQKGITSVWIPPAYKGTTQNDVGYGAYDLYDLGEFNQKGTVRTKYGTKAQLKSAIEALHKQNINVYGDVVMNHKGGADYTETVTAVEVDRNNRNIEVSGDYEINAWTGFNFPGRGDTHSNFKWKWYHFDGTDWDEGRKLNRIYKFRGIGKAWDWEVSSENGNYDYLMYADLDFDHPDVANEMKNWGTWYANELNLDGFRLDAVKHIDHEYLRDWVNHVRQQTGKEMFTVAEYWQNDIQTLNNYLAKVNYNQSVFDAPLHYNFHYASKGNGNYDMRNILNGTVMQNHPALAVTLVENHDSQPGQSLESVVSPWFKPLAYAFILTRAEGYPSVFYGDYYGTSGNSSYEIPALKDKIDPILMARKNFAYG

SEQ ID NO: 67 - protein, 30 aa, Bacillus cereus
  REGION 1..30: note = signal peptide AmyB
MKNQFQYCCIVILSVVMLFVSLLIPQASSA

SEQ ID NO: 68 - protein, 27 aa, Bacillus subtilis
  REGION 1..27: note = signal peptide AmyE
MFAKRFKTSLLPLFAGFLLLFHLVLAG

SEQ ID NO: 69 - protein, 29 aa, Bacillus licheniformis
  REGION 1..29: note = signal peptide AmyL
MKQQKRLYARLLTLLFALIFLLPHSAAAA

SEQ ID NO: 70 - protein, 33 aa, Bacillus stearothermophilus
  REGION 1..33: note = signal peptide AmyM
MKKKTLSLFVGLMLLIGLLFSGSLPYNPNAAEA

SEQ ID NO: 71 - protein, 31 aa, Bacillus amyloliquefaciens
  REGION 1..31: note = signal peptide AmyQ
MIQKRKRTVSFRLVLMCTLLFVSLPITKTSA

SEQ ID NO: 72 - protein, 34 aa, Bacillus stearothermophilus
  REGION 1..34: note = signal peptide AmyS
MLTFHRIIRKGWMFLLAFLLTALLFCPTGQPAKA

SEQ ID NO: 73 - protein, 29 aa, Bacillus subtilis
  REGION 1..29: note = signal peptide AprE
MRSKKLWISLLFALTLIFTMAFSNMSAQA

SEQ ID NO: 74 - protein, 27 aa, Bacillus licheniformis
  REGION 1..27: note = signal peptide AprE
MMRKKSFWLGMLTAFMLVFTMASIASA

SEQ ID NO: 75 - protein, 29 aa, Bacillus pumilus
  REGION 1..29: note = signal peptide AprE
MKKKNVMTSVLLAVPLLFSAGFGGSMAQA

SEQ ID NO: 76 - protein, 27 aa, Bacillus clausii
  REGION 1..27: note = signal peptide AprH
MKKPLGKIVASTALLISVAFSSSIASA

SEQ ID NO: 77 - protein, 22 aa, Bacillus subtilis
  REGION 1..22: note = signal peptide AspB
MKLAKRVSALTPSTTLAITAKA

SEQ ID NO: 78 - protein, 29 aa, Bacillus subtilis
  REGION 1..29: note = signal peptide BglC
MKRSISIFITCLLITLLTMGGMIASPASA

SEQ ID NO: 79 - protein, 28 aa, Bacillus subtilis
  REGION 1..28: note = signal peptide BglS
MPYLKRVLLLLVTGLFMSLFAVTATASA

SEQ ID NO: 80 - protein, 30 aa, Bacillus subtilis
  REGION 1..30: note = signal peptide Bpr
MRKKTKNRLISSVLSTVVISSLLFPGAAGA

SEQ ID NO: 81 - protein, 46 aa, Streptomyces lividans
  REGION 1..46: note = signal peptide CelA
MGFGSAPIALCPLRTRRNALKRLLALLATGVSIVGLTALAGPPAQA

SEQ ID NO: 82 - protein, 49 aa, Bacillus licheniformis
  REGION 1..49: note = signal peptide CelA
MMAEKVFSKNKIIGGKRMSYMKRSISVFIACFMVAVLGISGIIAPKASA

SEQ ID NO: 83 - protein, 35 aa, Bacillus subtilis
  REGION 1..35: note = signal peptide Csn
MKISMQKADFWKKAAISLLVFTMFFTLMMSETVFA

SEQ ID NO: 84 - protein, 27 aa, Bacillus subtilis
  REGION 1..27: note = signal peptide Epr
MKNMSCKLVVSVTLFFSFLTIGPLAHA

SEQ ID NO: 85 - protein, 41 aa, Bacillus licheniformis
  REGION 1..41: note = signal peptide ForD
MKNHLYEKKKRKPLTRTIKATLAVLTMSIALVGGATVPSLA

SEQ ID NO: 86 - protein, 25 aa, Bacillus licheniformis
  REGION 1..25: note = signal peptide GGT
MRRLAFLVVAFCLAVGCFFSPVSKA

SEQ ID NO: 87 - protein, 32 aa, Bifidobacterium bifidum
  REGION 1..32: note = signal peptide LacZ
MAVRRLGGRIVAFAATVALSIPLGLLTNSAWA

SEQ ID NO: 88 - protein, 31 aa, Bacillus subtilis
  REGION 1..31: note = signal peptide LipA
MKFVKRRIIALVTILMLSVTSLFALQPSAKA

SEQ ID NO: 89 - protein, 25 aa, Bacillus subtilis
  REGION 1..25: note = signal peptide LytB
MKSCKQLIVCSLAAILLLIPSVSFA

SEQ ID NO: 90 - protein, 27 aa, Bacillus subtilis
  REGION 1..27: note = signal peptide LytD
MKKRLIAPMLLSAASLAFFAMSGSAQA

SEQ ID NO: 91 - protein, 21 aa, Bacillus subtilis
  REGION 1..21: note = signal peptide Pel
MKKVMLATALFLGLTPAGANA

SEQ ID NO: 92 - protein, 51 aa, Bacillus subtilis
  REGION 1..51: note = signal peptide PhoD
MAYDSRFDEWVQKLKEESFQNNTFDRRKFIQGAGKIAGLSLGLTIAQSVGA

SEQ ID NO: 93 - protein, 19 aa, Bacillus subtilis
  REGION 1..19: note = signal peptide PhrK
MKKLVLCVSILAVILSGVA

SEQ ID NO: 94 - protein, 31 aa, Bacillus subtilis
  REGION 1..31: note = signal peptide Vpr
MKKGIIRFLLVSFVLFFALSTGITGVQAAPA

SEQ ID NO: 95 - protein, 32 aa, Bacillus subtilis
  REGION 1..32: note = signal peptide WapA
MKKRKRRNFKRFIAAFLVLALMISLVPADVLA

SEQ ID NO: 96 - protein, 25 aa, Bacillus subtilis
  REGION 1..25: note = signal peptide YbdN
MVKKWLIQFAVMLSVLSTFTYSASA

SEQ ID NO: 97 - protein, 26 aa, Bacillus subtilis
  REGION 1..26: note = signal peptide YckD
MKRITINIITMFIAAAVISLTGTAEA

SEQ ID NO: 98 - protein, 28 aa, Bacillus subtilis
  REGION 1..28: note = signal peptide YddT
MRKKRVITCVMAASLTLGSLLPAGYASA

SEQ ID NO: 99 - protein, 29 aa, Bacillus subtilis
  REGION 1..29: note = signal peptide YfhK
MKKKQVMLALTAAAGLGLTALHSAPAAKA

SEQ ID NO: 100 - protein, 21 aa, Bacillus subtilis
  REGION 1..21: note = signal peptide YfjS
MKWMCSICCAAVLLAGGAAQA

SEQ ID NO: 101 - protein, 34 aa, Bacillus subtilis
  REGION 1..34: note = signal peptide YhfM
MKKIVAAIVVIGLVFIAFFYLYSRSGDVYQSVDA

SEQ ID NO: 102 - protein, 28 aa, Bacillus subtilis
  REGION 1..28: note = signal peptide YjfA
MKRLFMKASLVLFAVVFVFAVKGAPAKA

SEQ ID NO: 103 - protein, 26 aa, Bacillus subtilis
  REGION 1..26: note = signal peptide YkwD
MKKAFILSAAAAVGLFTFGGVQQASA

SEQ ID NO: 104 - protein, 42 aa, Bacillus subtilis
  REGION 1..42: note = signal peptide YncM
MAKPLSKGGILVKKVLIAGAVGTAVLFGTLSSGIPGLPAADA

SEQ ID NO: 105 - protein, 32 aa, Bacillus subtilis
  REGION 1..32: note = signal peptide YnfF
MIPRIKKTICVLLVCFTMLSVMLGPGATEVLA

SEQ ID NO: 106 - protein, 25 aa, Bacillus subtilis
  REGION 1..25: note = signal peptide YobB
MKIRKILLSSALSFGMLISAVPALA

SEQ ID NO: 107 - protein, 30 aa, Bacillus subtilis
  REGION 1..30: note = signal peptide YvcE
MRKSLITLGLASVIGTSSFLIPFTSKTASA

SEQ ID NO: 108 - protein, 26 aa, Bacillus licheniformis
  REGION 1..26: note = signal peptide YvfO
MKNVLAVFVVLIFVLGAFGTSGPAEA

SEQ ID NO: 109 - protein, 44 aa, Bacillus subtilis
  REGION 1..44: note = signal peptide YwbN
MSDEQKKPEQIHRRDILKWGAMAGAAVAIGASGLGGLAPLVASA

(19) United States
(12) Patent Application Publication
(10) Pub. No.: US 2026/0028606 A1
(43) Pub. Date: Jan. 29, 2026

(54) USE OF FOLDASES TO IMPROVE HETEROLOGOUS EXPRESSION OF SECRETED MOLECULES

(71) Applicant: BASF SE, Ludwigshafen am Rhein (DE)

(72) Inventors: Christopher SAUER, Ludwigshafen am Rhein (DE); Stefan JENEWEIN, Ludwigshafen am Rhein (DE); Max Fabian FELLE, Ludwigshafen am Rhein (DE); Mathis APPELBAUM, Ludwigshafen am Rhein (DE)

(21) Appl. No.: 19/145,476

(22) PCT Filed: Jan. 4, 2024
(86) PCT No.: PCT/EP2024/050141

§ 371 Date: Jul. 2, 2025

(30) Foreign Application Priority Data
Jan. 5, 2023 (EP) 23150485.3

(51) Int. Cl. C12N 9/28; C12N 9/26; C12N 9/90; C12N 15/75

(52) U.S. Cl. CPC C12N 9/2417; C12N 9/2411; C12N 9/90; C12N 15/75; C12Y 302/01001; C12Y 302/01133; C12Y 502/01008; C12N 2800/101

(57) ABSTRACT

The present invention relates to a Bacillus licheniformis host cell for increased production of secreted proteins. Specifically, the invention relates to a Bacillus licheniformis host with a genetic modification that leads to increased production of a heterologous, non-native, secreted enzyme. Specifically, the present invention relates to a Bacillus licheniformis host cell expressing a) a first polypeptide having peptidyl-prolyl cis-trans isomerase (EC 5.2.1.8) activity, said first polypeptide comprising an amino acid sequence as shown in SEQ ID NO: 1, or an amino acid sequence being a least 81% identical to SEQ ID NO: 1, and b) a second polypeptide having amylase activity, wherein said second polypeptide is heterologous to said Bacillus licheniformis host cell.

### FIELD OF THE INVENTION

The present invention relates to a Bacillus licheniformis host cell for increased production of secreted proteins. Specifically, the invention relates to a Bacillus licheniformis host with a genetic modification that leads to increased production of a heterologous, non-native, secreted enzyme. Specifically, the present invention relates to a Bacillus licheniformis host cell expressing a) a first polypeptide having peptidyl-prolyl cis-trans isomerase (EC 5.2.1.8) activity, said first polypeptide comprising an amino acid sequence as shown in SEQ ID NO: 1, or an amino acid sequence being a least 81% identical to SEQ ID NO: 1, and b) a second polypeptide having amylase activity, wherein said second polypeptide is heterologous to said Bacillus licheniformis host cell.

### BACKGROUND

Microorganisms of the Bacillus genus are widely applied as industrial workhorses for the production of valuable compounds, such as chemicals, polymers and proteins, in particular proteins like washing- and/or cleaning-active enzymes or enzymes used for feed and food applications. The biotechnological production of these enzymes is conducted via fermentation of such Bacillus species and subsequent purification of the product. Bacillus species are capable of secreting significant amounts of protein to the fermentation broth. This allows a simple product purification process compared to intracellular production and explains the success of Bacillus in industrial application. Thus, the continuous optimization of Bacillus cells for increased production of these enzymes is of high relevance. In particular, in large-scale industrial production settings, even small improvements have a great impact on production costs.

Extracellular proteins and enzymes are secreted by Bacillus cells. The major route for protein transport across the cell membrane is the general secretion ‘Sec’ (SecA-YEG) pathway, where the unfolded polypeptide chain is translocated through the SecYEG translocase complex into the cell-wall associated space where the signal peptide is cleaved and folding of the polypeptide takes place.

The essential extra-cytoplasmic foldase PrsA is a lipoprotein with peptidyl-prolyl cis-trans isomerase activity assisting folding of the polypeptide into a stable mature protein conformation. The expression of prsA is feedback regulated by the secretion stress response and the PrsA foldase itself is substrate of multiple extracytoplasmic proteases (Krishnappa L, Monteferrante C G, Neef J, Dreisbach A, van Dijl J M. Degradation of extracytoplasmic cata-lysts for protein folding in Bacillus subtilis. Appl Environ Microbiol. 2014 February; 80(4):1463-8. doi: 10.1128/AEM.02799-13. Epub 2013 Dec. 20. PMID: 24362423; PMCID: PMC3911040.).

The additional expression of PrsA foldases native to the host has been shown to improve production of secreted proteins such as heterologous subtilisin proteases, lipases and especially amylases in Bacillus subtilis (WO94/019471) or Bacillus licheniformis (WO2021/146411).

WO2020/156903 discloses that both the PrsA protein and the amylase should be from the same species and at least some of such cognate combinations show improvement of amylase expression in Bacillus subtilis. This implies that PrsA foldases are in particular beneficial for improved production of polypeptides from the same species.

Identification of such a cognate PrsA protein and Amylase combination is not necessarily given. EP 1 307 547 A1 discloses an Amylase from a Bacillus species A7-7 (DSM 12368) with unknown genome sequence. Bacillus cereus ATCC14579 comprises three prsA genes (genome accession number AE016877), hence it is not obvious which PrsA foldase would be the optimal combination for improved expression of an amylase from Bacillus cereus.

In Bacillus pumilus SAFR-032, one prsA gene is encoded on the chromosome (Genbank accession number CP000813.4, Stepanov V G, Tirumalai M R, Montazari S, Checinska A, Venkateswaran K, Fox G E. Bacillus pumilus SAFR-032 Genome Revisited: Sequence Update and Re-Annotation. PLoS One. 2016 Jun. 28; 11(6):e0157331. doi: 10.1371/journal.pone.0157331. PMID: 27351589; PMCID: PMC4924849).

Also, amylases have been engineered to meet application-related functionalities such improved stability at higher temperature, within formulations such as detergents, denaturing conditions such as pH, or stability against proteases, hence the sequence of the Amylase deviating from the native sequence. The concept of cognate PrsA-Amylase combinations of WO2020/156903 can also not be applied to chimeric amylases, e.g. hybrid amylases where different domains of the protein are derived from different species.

The improvement of the above-mentioned product performance with engineered polypeptide variants however does not necessarily come along with optimal expression of such non-native proteins.

Therefore, there remains the need for host systems leading to overall enhanced production of polypeptides of interest, in particular for amylases.

### BRIEF SUMMARY OF THE INVENTION

Advantageously, it has been found in the studies underlying the present invention that a Bacillus licheniformis host cell expressing the prsA foldase (SEQ ID NO: 1, herein also referred to as “peptidyl-prolyl cis-trans isomerase”) from Bacillus pumilus results in increased production of a polypeptide of interest as compared to a control cell. Specifically, it was shown that the expression of amylases having an amino acid sequence as shown in SEQ ID NO: 29, 35, 38, 40, 42, 48, 50, 53, 59 or 61, or variants thereof, could be increased by co-expressing the PrsA foldase from Bacillus pumilus in a Bacillus licheniformis host cell. The effect is surprising since Bacillus pumilus SAFR-032, i.e. the organism from which the prsA foldase is derived, does not naturally comprise amylases. Notably, the production enhancing effect of the Bacillus pumilus PrsA foldase was only seen in Bacillus licheniformis cells, but not in Bacillus subtilis cells.

Accordingly the present invention relates to a Bacillus licheniformis host cell expressing
a) a first polypeptide having peptidyl-prolyl cis-trans isomerase (EC 5.2.1.8) activity, said first polypeptide comprising an amino acid sequence as shown in SEQ ID NO: 1, or an amino acid sequence being a least 81% identical to SEQ ID NO: 1, and
b) a second polypeptide having amylase activity, wherein said second polypeptide is heterologous to said Bacillus licheniformis host cell.

The present invention further relates to a method of producing a polypeptide having amylase activity, comprising
a) providing the Bacillus licheniformis host cell of the present invention, and
b) cultivating the Bacillus licheniformis host cell under conditions that allow for expressing said polypeptide having amylase activity, and optionally,
c) obtaining or purifying said polypeptide having amylase activity.

The present invention further relates to a method of producing the Bacillus licheniformis host cell of the present invention, comprising
a) providing a Bacillus licheniformis host cell, and
b) introducing into the host cell provided in step a)
   b1) a first polynucleotide encoding a first polypeptide having peptidyl-prolyl cis-trans isomerase (EC 5.2.1.8) activity, said first polypeptide comprising an amino acid sequence as shown in SEQ ID NO: 1, or an amino acid sequence being a least 81% identical to SEQ ID NO: 1, and
   b2) a second polynucleotide encoding a second polypeptide having amylase activity.

The present invention further relates to the use of
i) a first polypeptide having peptidyl-prolyl cis-trans isomerase (EC 5.2.1.8) activity, said first polypeptide comprising an amino acid sequence as shown in SEQ ID NO: 1, or an amino acid sequence being a least 81% identical to SEQ ID NO: 1, and/or
ii) a polynucleotide encoding said first polypeptide

for increasing the production of a second polypeptide having alpha amylase activity in a Bacillus licheniformis host cell.

Further, the present invention relates to the use of the Bacillus licheniformis host cell of the present invention for producing the second polypeptide, such as a polypeptide having alpha amylase activity.

In an embodiment of the host cell, of the methods and of the use of the present invention, the first polypeptide comprises an amino acid sequence being at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% or at least 100% identical to SEQ ID NO: 1.

In a preferred embodiment of the host cell, of the methods and of the use of the present invention, the second polypeptide has alpha amylase activity (EC 3.2.1.1).

In another preferred embodiment of the host cell, of the methods and of the use of the present invention, the second polypeptide has maltogenic alpha-amylase activity (EC 3.2.1.133).

In a preferred embodiment of the host cell, of the methods and of the use of the present invention, said second polypeptide comprises an amino acid sequence being at least 86%, at least 87%, at least 88%, at least 89%, at least 90%, at least 91%, at least 92%, at least 93%, at least 94%, at least 95%, at least 96%, at least 97%, at least 98%, at least 99% or at least 99.5% identical to SEQ ID NO: 29, 35, 38, 40, 42, 48, 50, 53, 59 or 61.

In a preferred embodiment of the host cell, of the methods and of the use of the present invention, said second polypeptide comprises an amino acid sequence as shown in SEQ ID NO: 29, 35, 38, 40, 42, 48, 50, 53, 59 or 61.

In a preferred embodiment of the host cell, of the methods and of the use of the present invention, said second polypeptide, e.g. the amylase, is secreted. Thus, the second polypeptide is a secreted polypeptide. Accordingly, the second polypeptide typically comprises a signal peptide, preferably, at the N-terminus.

In a preferred embodiment of the host cell, of the methods and of the use of the present invention, the host cell comprises:
a) a first expression cassette for said first polypeptide, said first expression cassette comprising a first promoter, operably linked to a first polynucleotide encoding said first polypeptide, and optionally a terminator, and
b) a second expression cassette for second polypeptide, said second expression cassette comprising a second promoter, operably linked to a second polynucleotide encoding said second polypeptide, and optionally a terminator.

In a preferred embodiment of the host cell, of the methods and of the use of the present invention, the first and/or second promoter is an inducer independent promoter, such as a constitutive promoter, for example, the promoter of a Bacillus aprE gene, such as the promoter of the Bacillus licheniformis aprE gene. In an embodiment, the promoter comprises a nucleic acid sequence as shown in SEQ ID NO: 3, or a variant of said promoter having at least 80%, at least 85%, at least 90%, at least 93%, at least 95%, at least 98%, at least 99%, or 100% sequence identity to SEQ ID NO: 3.

In one embodiment, both the first promoter and the second promoter are inducer independent promoters, such as constitutive promoters.

In another preferred embodiment of the host cell, of the methods and of the use of the present invention, the first and/or second promoter is an inducible promoter.

In an embodiment of the host cell, of the methods and of the use of the present invention, the first expression cassette and/or second expression cassette is (are) present in the host cell on a plasmid or is (are) stably integrated into the chromosomal DNA of the host cell.

Typically, the host cell is from a Bacillus licheniformis strain selected from the group consisting of Bacillus licheniformis strains ATCC 14580, ATCC 31972, ATCC 53757, ATCC 53926, ATCC 55768, DSM 13, DSM 394, DSM 641, DSM 1913, DSM 11259, and DSM 26543.

### DETAILED DESCRIPTION OF THE INVENTION—DEFINITIONS

It is to be understood that as used in the specification and in the claims, “a” or “an” can mean one or more, depending upon the context in which it is used. Thus, for example, reference to “a cell” can mean that at least one cell can be utilized.

Further, it will be understood that the term “at least one” as used herein means that one or more of the items referred to following the term may be used in accordance with the invention. For example, if the term indicates that at least one feed solution shall be used this may be understood as one feed solution or more than one feed solutions, i.e. two, three, four, five or any other number of feed solutions. Depending on the item the term refers to the skilled person understands as to what upper limit the term may refer, if any.

The term “about” as used herein means that with respect to any number recited after said term an interval accuracy exists within in which a technical effect can be achieved. Accordingly, about as referred to herein, preferably, refers to the precise numerical value or a range around said precise numerical value of ±20%, preferably ±15%, more preferably ±10%, and even more preferably ±5%.

The term “comprising” as used herein shall not be understood in a limiting sense. The term rather indicates that more than the actual items referred to may be present, e.g., if it refers to a method comprising certain steps, the presence of further steps shall not be excluded. However, the term “comprising” also encompasses embodiments where only the items referred to are present, i.e. it has a limiting meaning in the sense of “consisting of”.

The terms “polynucleotide”, “nucleic acid sequence”, “nucleotide sequence”, “nucleic acid”, “nucleic acid molecule” are used interchangeably herein and refer to nucleotides, typically deoxyribonucleotides, in a polymeric unbranched form of any length. The terms “polypeptide” and “protein” are used interchangeably herein and refer to amino acids in a polymeric form of any length, linked together by peptide bonds.

The terms “coding for” and “encoding” are used interchangeably herein. Typically, the terms refer to the property of specific sequences of nucleotides in a polynucleotide, such as a gene, a cDNA, or an mRNA, to serve as templates for synthesis of other macromolecules such as a defined sequence of amino acids. Thus, a gene codes for a protein, if transcription and translation of mRNA corresponding to that gene produces the protein in a cell or other biological system.

In accordance with the present invention, the first and the second polypeptide, as well as variants thereof, as defined elsewhere herein shall be expressed in the host cell.

Variants of a parent molecule may have an amino acid sequence which is at least n percent identical to the amino acid sequence of the respective parent enzyme having enzymatic activity with n being an integer between 50 and 100, preferably 50, 55, 60, 65, 70, 75, 80, 85, 90, 91, 92, 93, 94, 95, 96, 97, 98 or 99 compared to the full length polypeptide sequence. Variant enzymes described herein which are n percent identical when compared to a parent enzyme have enzymatic activity.

In some embodiments, a variant of a parent polypeptide comprises an amino acid sequence which is at least 50, at least 60%, at least 70%, at least 80%, at least 85%, at least 90%, at least 95% or at least 98%, but below 100% identical to an amino acid sequence of the parent polypeptide.

Variants may be, thus, defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment).

The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

After aligning the two sequences, in a second step, an identity value shall be determined from the alignment. Therefore, according to the present invention the following calculation of percent-identity applies:

%-identity=(identical residues/length of the alignment region which is showing the respective sequence of this invention over its complete length)*100.

Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”.

For calculating the percent identity of two DNA sequences the same applies as for the calculation of percent identity of two amino acid sequences with some specifications. For DNA sequences encoding a protein the pairwise alignment shall be made over the complete length of the coding region from start to stop codon excluding introns. For non-protein-coding DNA sequences the pairwise alignment shall be made over the complete length of the sequence of this invention, so the complete sequence of this invention is compared to another sequence, or regions out of another sequence. Moreover, the preferred alignment program implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

Variant polypeptides may also be defined by their sequence similarity when compared to another sequence. Sequence similarity usually is provided as “% sequence similarity” or “%-similarity”. For calculating sequence similarity in a first step a sequence alignment has to be generated as described above. In a second step, the percent-similarity has to be calculated, whereas percent sequence similarity takes into account that defined sets of amino acids share similar properties, e.g., by their size, by their hydrophobicity, by their charge, or by other characteristics. Herein, the exchange of one amino acid with a similar amino acid is referred to as “conservative mutation”. Enzyme variants comprising conservative mutations appear to have a minimal effect on protein folding resulting in certain enzyme properties being substantially maintained when compared to the enzyme properties of the parent enzyme.

For determination of %-similarity according to this invention the following applies, which is also in accordance with the BLOSUM62 matrix, which is one of the most used amino acids similarity matrix for database searching and sequence alignments

Amino acid A is similar to amino acid S
Amino acid D is similar to amino acids E; N
Amino acid E is similar to amino acids D; K; Q
Amino acid F is similar to amino acids W; Y
Amino acid H is similar to amino acids N; Y
Amino acid I is similar to amino acids L; M; V
Amino acid K is similar to amino acids E; Q; R
Amino acid L is similar to amino acids I; M; V
Amino acid M is similar to amino acids I; L; V
Amino acid N is similar to amino acids D; H; S
Amino acid Q is similar to amino acids E; K; R
Amino acid R is similar to amino acids K; Q
Amino acid S is similar to amino acids A; N; T
Amino acid T is similar to amino acid S
Amino acid V is similar to amino acids I; L; M
Amino acid W is similar to amino acids F; Y
Amino acid Y is similar to amino acids F; H; W.

Conservative amino acid substitutions may occur over the full length of the sequence of a polypeptide sequence of a functional protein such as an enzyme. In one embodiment, such mutations are not pertaining to the functional domains of an enzyme. In another embodiment conservative mutations are not pertaining to the catalytic centers of an enzyme.

Therefore, the following calculation of percent-similarity applies:

%-similarity=[(identical residues+similar residues)/length of the alignment region which is showing the respective sequence of this invention over its complete length]*100.

Thus, sequence similarity in relation to comparison of two amino acid sequences herein is calculated by dividing the number of identical residues plus the number of similar residues by the length of the alignment region which is showing the respective sequence of this invention over its complete length. This value is multiplied by 100 to give “%-similarity”.

Especially, variant enzymes comprising conservative mutations which are at least m percent similar to the respective parent sequences with m being an integer between 50 and 100, preferably 50, 55, 60, 65, 70, 75, 80, 85, 90, 91, 92, 93, 94, 95, 96, 97, 98 or 99 compared to the full length polypeptide sequence, are expected to have essentially unchanged enzyme properties.

A variant of a parent polypeptide may have the activity or function of the parent enzyme. Thus, a variant of an amylase shall have amylase activity, typically in the same EC class. Variant enzymes described herein with m percent-similarity when compared to a parent enzyme, thus have enzymatic activity.

“Variant” enzymes differ from “parent” enzymes by certain amino acid alterations, preferably amino acid substitutions at one or more amino acid positions.

In describing the polypeptide variants, the abbreviations for single amino acids are used according to the accepted IUPAC single letter or three letter amino acid abbreviations.

“Amino acid alteration” as used herein refers to amino acid substitution, deletion, or insertion.

“Substitutions” are described by providing the original amino acid followed by the number of the position within the amino acid sequence, followed by the amino acid, which substitutes the original amino acid. For example, the substitution of histidine at position 120 with alanine is designated as “His120Ala” or “H120A”. Substitutions can also be described by merely naming the resulting amino acid in the variant without specifying the amino acid of the parent at this position, e.g., “X120A” or “120A” or “Xaa120Ala” or “120Ala”.

“Deletions” are described by providing the original amino acid followed by the number of the position within the amino acid sequence, followed by *. Accordingly, the deletion of glycine at position 150 is designated as “Gly150*” or G150*”. Alternatively, deletions are indicated by, e.g., “deletion of D183 and G184”.

“Insertions” are described by providing the original amino acid followed by the number of the position within the amino acid sequence, followed by the original amino acid and the additional amino acid. For example, an insertion at position 180 of lysine next to glycine is designated as “Gly180GlyLys” or “G180GK”. When more than one amino acid residue is inserted, such as, e.g., a Lys and an Ala after Gly180 this may be indicated as: “Gly180GlyLysAla” or “G195GKA”.

In cases where a substitution and an insertion occur at the same position, this may be indicated as “S99SD+S99A” or in short “S99AD”. Variants comprising multiple alterations are separated by “+”, e.g., “Arg170Tyr+Gly195Glu”, “R170Y+G195E” or “X170Y+X195E” representing a substitution of arginine and glycine at positions 170 and 195 with tyrosine and glutamic acid, respectively. Alternatively, multiple alterations may be separated by space or a comma, e.g., “R170Y G195E” or “R170Y, G195E” respectively. Where different alternative alterations can be introduced at a position, the different alterations are separated by a comma, e.g., “Arg170Tyr, Glu” and “R170T, E”, respectively, represents a substitution of arginine at position 170 with tyrosine or glutamic acid. Alternative substitutions at a particular position can also be indicated as “X120A,G,H”, “120A,G,H”, “X120A/G/H”, or “120A/G/H”. Alternatively, different alterations or optional substitutions may be indicated in brackets, e.g., “Arg170[Tyr, Gly]” or “Arg170{Tyr, Gly}” or in short “R170 [Y, G]” or “R170 {Y, G}”.

### The Host Cell

The Bacillus licheniformis host cell of the present invention comprises
a) a first polypeptide having peptidyl-prolyl cis-trans isomerase (EC 5.2.1.8) activity as defined elsewhere herein, and
b) a second polypeptide which is heterologous to said Bacillus licheniformis host cell, for example an amylase.

Accordingly, the Bacillus licheniformis host cell of the present invention, preferably, comprises
a) a first polynucleotide encoding the first polypeptide having peptidyl-prolyl cis-trans isomerase (EC 5.2.1.8) activity, and
b) a second polynucleotide encoding the second polypeptide which is heterologous to said Bacillus licheniformis host cell, for example an amylase.

Preferably, the first and the second polynucleotide are comprised by an expression cassette. Accordingly, the Bacillus licheniformis host cell of the present invention comprises
a) a first expression cassette for said first polypeptide, said first expression cassette comprising a first promoter, operably linked to a first polynucleotide encoding said first polypeptide, and optionally a terminator, and
b) a second expression cassette for said second polypeptide, said second expression cassette comprising a second promoter, operably linked to a second polynucleotide encoding said second polypeptide, and optionally a terminator.

The term “host cell” in accordance with the present invention is a Bacillus licheniformis host cell. Preferably, the Bacillus licheniformis host cell is of the strain Bacillus licheniformis ATCC 14580, ATCC 31972, ATCC 53757, ATCC 53926, ATCC 55768, DSM 13, DSM 394, DSM 641, DSM 1913, DSM 11259 or DSM 26543. In an embodiment, the host cell is of the Bacillus licheniformis strain as deposited under American Type Culture Collection number ATCC14580 (which is the same as DSM13, see Veith et al. “The complete genome sequence of Bacillus licheniformis DSM13, an organism with great industrial potential.” J. Mol. Microbiol. Biotechnol. (2004) 7:204-211). Alternatively, the host cell is a host cell of Bacillus licheniformis strain ATCC31972. Alternatively, the host cell is a host cell of Bacillus licheniformis strain ATCC53757. Alternatively, the host cell is a host cell of Bacillus licheniformis strain ATCC53926. Alternatively, the host cell is a host cell of Bacillus licheniformis strain ATCC55768. Alternatively, the host cell is a host cell of Bacillus licheniformis strain DSM394. Alternatively, the host cell is a host cell of Bacillus licheniformis strain DSM641. Alternatively, the host cell is a host cell of Bacillus licheniformis strain DSM1913. Alternatively, the host cell is a host cell of Bacillus licheniformis strain DSM11259. Alternatively, the host cell is a host cell of Bacillus licheniformis strain DSM26543.

Furthermore, it is envisaged that the modified host cell as set forth herein does not produce poly-gamma-glutamate (pga) or produces a reduced amount of pga. Accordingly, at least one gene involved in poly-gamma-glutamate (pga) production has been inactivated (such as deleted). Preferably, the at least one gene involved in poly-gamma-glutamate (pga) is at least one gene selected from ywsC (pgsB), ywtA (pgsC), ywtB (pgsA) and ywtC (pgsE). Preferably, all aforementioned genes, i.e. ywsC (pgsB), ywtA (pgsC), ywtB (pgsA) and ywtC (pgsE) have been inactivated (such as deleted).

Furthermore, it is envisaged that the modified host cell is not capable to sporulate. This may be achieved by inactivating (such as deleting) at least one gene involved in sporulation. Genes involved in sporulation are well known in the art (EP1391502), comprising but not limited to sigE, sigF, spoIIGA, spoIIE, sigG, spoIVCB, yqfD. In a preferred embodiment, the sigF gene is deleted.

Furthermore, it is envisaged that the modified host cell is reduced in proteolytic activities (as compared to a control cell). This can be achieved by inactivating (such as deleting) at least one protease encoding gene comprising but not limited to aprE, mpr, bpr, vpr, epr, wprA, ispA, aprX. Preferably the aprE and the mpr genes are deleted, most preferably the aprE gene is deleted.

Furthermore, it is envisaged that the modified host cell is reduced in glycosidase activities. This can be achieved by inactivating (such as deleting) at least one gene encoding glycosidases comprising but not limited to alpha-amylase (EC 3.2.1.1), beta amylase (EC 3.2.1.2) and glucan 1,4-alpha-maltohydrolase (EC 3.2.1.133)), a cellulase (EC 3.2.1.4), an endo-1,3-beta-xylanase xylanase (EC 3.2.1.32), an endo-1,4-beta-xylanase (EC 3.2.1.8), a lactase (EC 3.2.1.108), a galactosidase (EC 3.2.1.23 and EC 3.2.1.24), a mannanase (EC 3.2.1.24 and EC 3.2.1.25).

In a preferred embodiment, the gene encoding the endogenous alpha-amylase polypeptide (as shown in SEQ ID NO: 35) is deleted.

### First Polypeptide (Peptidyl-Prolyl Cis-Trans Isomerase)

The first polypeptide as referred to herein has peptidyl-prolyl cis-trans isomerase activity (EC 5.2.1.8). Thus, the first polypeptide is a peptidyl-prolyl cis-trans isomerase (frequently also referred to as “foldase”, “peptidylprolyl isomerase”, “peptide bond isomerase” or “PPlase”). The terms “foldase”, “peptidyl-prolyl cis-trans isomerase” and “PrsA” are used interchangeably herein.

As used herein, the term “peptidyl-prolyl cis-trans isomerase” refers to an enzyme that inter-converts the cis and trans isomers of peptide bonds with the amino acid proline. Thus, it interconverts the cis and trans isomers of peptidyl-prolyl bonds within a protein. In Bacillus, peptidyl-prolyl cis-trans isomerase that are membrane-bound lipoproteins that are assumed to assist post-translocational folding of secreted proteins and stabilize them in the compartment between the cytoplasmic membrane and cell wall.

Typically, the active form of the enzyme is a dimer of two monomers, i.e. a dimer formed by two monomers of the first polypeptide. Thus, it is understood by the skilled person that it is the dimer that has peptidyl-prolyl cis-trans isomerase activity. Typically, the peptidyl-prolyl cis-trans isomerase has two domains, a peptidyl-prolyl cis-trans isomerase (EC 5.2.1.8) domain and a chaperone domain which supports protein folding.

In accordance with the present invention, the first polypeptide is the PrsA protein (SEQ ID NO: 1) from B. pumilis or a variant thereof. Preferably, said first polypeptide comprises an amino acid sequence as shown in SEQ ID NO: 1, or an amino acid sequence being a least 81% identical to SEQ ID NO: 1. More preferably, the first polypeptide comprises an amino acid sequence being at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to SEQ ID NO: 1. Moreover, the first polypeptide may comprise or consist of an amino acid sequence as shown in SEQ ID NO: 1.

Further, the first polypeptide (i.e. the dimer formed by two monomers of said first polypeptide), preferably, has peptidyl-prolyl cis-trans isomerase activity. Whether a polypeptide has this activity can be assessed by well-known assays, e.g. by the assay as described in Jakob et al., 2009 (Proc Natl Acad Sci USA. 2009 Dec. 1; 106(48):20282-7. doi: 10.1073/pnas.0909544106. Epub 2009 Nov. 17. PMID: 19920179; PMCID: PMC2787138), and in Jakob et al. 2014(J Biol Chem. 2015 Feb. 6; 290(6):3278-92. doi: 10.1074/jbc.M114.622910. Epub 2014 Dec. 17. PMID: 25525259; PMCID: PMC4319002). Also preferably, the first polypeptide is capable of being transported and being bound to the cell membrane of the host cell. Thus, first polypeptide comprises a suitable signal peptide, such as the signal peptide of the native enzyme.

Thus, the first polypeptide is bound to the cell membrane of the host cell, i.e. it is present in the host cell as membrane-bound polypeptide.

In an embodiment, the variant of polypeptide has at least 50%, 60%, 70%, 80%, 85%, 90%, 95% or 98% peptidyl-prolyl cis-trans isomerase activity of the parent polypeptide (i.e. the polypeptide having a sequence as shown in SEQ ID NO:2).

In accordance with the present invention, the first polypeptide assists the folding of the second polypeptide which is co-expressed with the first polypeptide in the host, e.g. of an amylase.

In one embodiment of the present invention, the host cell expresses the endogenous PrsA polypeptide, i.e. the Bacillus licheniformis PrsA polypeptide. The amino acid sequence of the Bacillus licheniformis PrsA polypeptide is shown in SED ID NO: 12.

In an alternative embodiment of the present invention, the host cell does not express the endogenous PrsA polypeptide. Thus, the endogenous prsA gene is deleted. The nucleic acid sequence of the endogenous prsA gene is shown in SED ID NO: 13.

### Second Polypeptide (“Polypeptide of Interest”)

The second polypeptide is herein also referred to as “polypeptide of interest”. The terms are used interchangeably herein.

Preferably, the second polypeptide is an amylase, i.e. a polypeptide having amylase activity. The amylase may be a naturally occurring amylase or a non-naturally occurring amylase.

The term “amylase” as used herein typically refers to an enzyme having “amylolytic activity” or “amylase activity”. “Amylolytic activity” or “amylase activity” describes the capability for the hydrolysis of glucosidic linkages in polysaccharides. Amylase activity may be determined by assays for measurement of amylase activity which are known to those skilled in the art. Examples for assays measuring amylase activity are the Phadebas assay or the EPS assay (“Infinity reagent”). In the Phadebas assay amylase activity is determined by employing Phadebas tablets as substrate (Phadebas Amylase Test, supplied by Magle Life Science). Starch is hydrolyzed by the amylase giving soluble blue fragments. The absorbance of the resulting blue solution, measured spectrophotometrically at 620 nm, is a function of the amylase activity. The measured absorbance is directly proportional to the specific activity (activity/mg of pure amylase protein) of the amylase in question under the given set of conditions.

Alternatively, amylase activity can also be determined by a method employing the Ethyliden-4-nitrophenyl-alpha-D-maltoheptaosid (EPS). D-maltoheptaoside is a blocked oligosaccharide which can be cleaved by an endo-amylase. Following the cleavage, the alpha-glucosidase included in the kit to digest the substrate to liberate a free PNP molecule which has a yellow color and thus can be measured by visible spectophotometry at 405 nm. Kits containing EPS substrate and alpha-glucosidase is manufactured for example by Roche Costum Biotech (cat. No. 10880078103). The slope of the time dependent absorption-curve is directly proportional to the specific activity (activity per mg enzyme) of the amylase in question under the given set of conditions.

Typically, an amylase as referred herein is an alpha amylase (EC 3.2.1.1), a beta amylase (EC 3.2.1.2) or maltogenic alpha amylase (EC 3.2.1.133).

In a preferred embodiment, the amylase is an alpha-amylase (EC 3.2.1.1). An alpha-amylase is an enzyme that catalyzes the endohydrolysis of (1-4)-α-D-glucosidic linkages in polysaccharides containing three or more (1-4)-α-linked D-glucose units. The enzyme acts, e.g. on starch or glycogen in a random manner; reducing groups are liberated in the alpha-configuration, i.e. the initial anomeric configuration of the free sugar group released. Other names are glycogenase, endoamylase, 4-α-D-glucan glucanohydrolase and 1,4-α-D-glucan glucanohydrolase. The systematic name is “4-α-D-glucan glucanohydrolase”. For example, the polypeptides with an amino acid sequence as shown in SEQ ID NO: 29, 35, 38, 40, 42, 48, 50, 59 and 61, respectively, have alpha-amylase activity (EC 3.2.1.1). Variants of these amylase shall have the alpha-amylase activity as well.

In another preferred embodiment, the amylase is a beta-amylase (EC 3.2.1.2). A beta-amylase is an enzyme that catalyzes the hydrolysis of (1-4)-α-D-glucosidic linkages in polysaccharides so as to remove successive maltose units from the non-reducing ends of the chains. The enzyme acts, e.g. on starch or glycogen producing #-maltose by an inversion. Other names are saccharogen amylase, glycogenase, #amylase or 1,4-α-D-glucan maltohydrolase. The systematic name is “1,4-α-D-glucan maltohydrolase”.

In another preferred embodiment, the amylase is a maltogenic alpha-amylase (EC 3.2.1.133). A maltogenic alpha-amylase (herein also referred to as “glucan 1,4-α-maltohydrolase”) is an enzyme that catalyzes the hydrolysis of (1-4)-α-D-glucosidic linkages in polysaccharides so as to remove successive α-maltose residues from the non-reducing ends of the chains. The enzyme acts, e.g. on starch and related polysaccharides and oligosaccharides. The product is α-maltose. Other names are glucan 1,4-α-maltohydrolase or 1,4-α-D-glucan α-maltohydrolase. The systematic name is “1,4-α-D-glucan α-maltohydrolase”. For example, the polypeptide with an amino acid sequence as shown in SEQ ID NO: 53 has maltogenic alpha-amylase activity (EC 3.2.1.133). Variants of this amylase shall have the maltogenic alpha-amylase activity as well.

In a preferred embodiment, the amylase as referred to herein comprises an amino acid sequence being at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% identical to the amino acid sequence as shown in SEQ ID NO: 29, 35, 38, 40, 42, 48, 50, 53, 59 or 61.

For example, the amylase as referred to herein comprises an amino acid sequence being at least 95%, at least 96%, at least 97%, at least 98%, at least 99%, at least 99.5% or 100% identical to the amino acid sequence as shown in SEQ ID NO: 29, 35, 38, 40, 42, 48, 50, 53, 59 or 61.

For example, the amylase as referred to herein comprises an amino acid sequence as shown in SEQ ID NO: 29, 35, 38, 40, 42, 48, 50, 53, 59 or 61.

In an embodiment, the amylase as referred to herein comprises an amino acid sequence being at least 86%, at least 87%, at least 88%, at least 89%, at least 90%, at least 91%, at least 92%, at least 93%, at least 94%, at least 95%, at least 96%, at least 97%, at least 98%, at least 99% or at least 99.5% or 100% identical to SEQ ID NO: 29.

In another embodiment, the amylase as referred to herein comprises an amino acid sequence being at least 86%, at least 87%, at least 88%, at least 89%, at least 90%, at least 91%, at least 92%, at least 93%, at least 94%, at least 95%, at least 96%, at least 97% identical to SEQ ID NO: 35. In some embodiments, said amylase has less than 99% sequence identity, such as less than 98% or less than 97% sequence identity to SEQ ID NO:35.

In yet another embodiment, the amylase as referred to herein comprises an amino acid sequence being at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% identical to SEQ ID NO: 38.

In yet another embodiment, the amylase as referred to herein comprises an amino acid sequence being at least 86%, at least 87%, at least 88%, at least 89%, at least 90%, at least 91%, at least 92%, at least 93%, at least 94%, at least 95%, at least 96%, at least 97%, at least 98%, at least 99% or at least 99.5% or 100% identical to SEQ ID NO: 40.

In yet another embodiment, the amylase as referred to herein comprises an amino acid sequence being at least 86%, at least 87%, at least 88%, at least 89%, at least 90%, at least 91%, at least 92%, at least 93%, at least 94%, at least 95%, at least 96%, at least 97%, at least 98%, at least 99% or at least 99.5% or 100% identical to SEQ ID NO: 42.

In yet another embodiment, the amylase as referred to herein comprises an amino acid sequence being at least 86%, at least 87%, at least 88%, at least 89%, at least 90%, at least 91%, at least 92%, at least 93%, at least 94%, at least 95%, at least 96%, at least 97%, at least 98%, at least 99% or at least 99.5% or 100% identical to SEQ ID NO: 48.

In yet another embodiment, the amylase as referred to herein comprises an amino acid sequence being at least 86%, at least 87%, at least 88%, at least 89%, at least 90%, at least 91%, at least 92%, at least 93%, at least 94%, at least 95%, at least 96%, at least 97%, at least 98%, at least 99% or at least 99.5% or 100% identical to SEQ ID NO: 50.

In yet another embodiment, the amylase as referred to herein comprises an amino acid sequence being at least 86%, at least 87%, at least 88%, at least 89%, at least 90%, at least 91%, at least 92%, at least 93%, at least 94%, at least 95%, at least 96%, at least 97%, at least 98%, at least 99% or at least 99.5% or 100% identical to SEQ ID NO: 59.

In yet another embodiment, the amylase as referred to herein comprises an amino acid sequence being at least 86%, at least 87%, at least 88%, at least 89%, at least 90%, at least 91%, at least 92%, at least 93%, at least 94%, at least 95%, at least 96%, at least 97%, at least 98%, at least 99% or at least 99.5% or 100% identical to SEQ ID NO: 61.

In yet another embodiment, the amylase as referred to herein comprises an amino acid sequence being at least 86%, at least 87%, at least 88%, at least 89%, at least 90%, at least 91%, at least 92%, at least 93%, at least 94%, at least 95%, at least 96%, at least 97%, at least 98%, at least 99% or at least 99.5% identical to SEQ ID NO: 53.

As set forth above, the amylase may comprise an amino acid sequence as shown in SEQ ID NO: 35 (or is a variant thereof). The amylase with SEQ ID NO: 35 is the amylase from Bacillus licheniformis. This amylase has been described (as SEQ ID NO:2) as described in WO 95/10603. Suitable variants that could be used in the context of the present invention are described in WO 95/10603 comprising one or more substitutions in the following positions: 15, 23, 105, 106, 124, 128, 133, 154, 156, 178, 179, 181, 188, 190, 197, 201, 202, 207, 208, 209, 211, 243, 264, 304, 305, 391, 408, and 444 which have amylolytic activity. Variants are described in WO 94/02597, WO 94/018314, WO 97/043424 and SEQ ID NO:4 of WO 99/019467.

As set forth above, the amylase may comprise an amino acid sequence as shown in SEQ ID NO: 59 (or is a variant thereof). The amylase with SEQ ID NO: 59 is the amylase from Bacillus halmapalus, also referred to as “SP-722 amylase”. It also has been described as SEQ ID NO:2 or SEQ ID NO:7 in WO 96/23872. Preferred variants that could be used in the context of the present invention are described in WO 97/3296, WO 99/194671 and WO 2013/001078.

As set forth above, the amylase may comprise an amino acid sequence as shown in SEQ ID NO: 38 (or is a variant thereof). The amylase with SEQ ID NO:38 is the amylase from Bacillus sp. A 7-7 (DSM 12368). In an embodiment, the amylase comprises an amino acid sequence at least 95% identical to SEQ ID NO:2, in particular over the region of the amino acids 32 to 516 according to SEQ ID NO:2, as disclosed in WO 02/10356. SEQ ID NO:2 as disclosed in WO02/10356 is identical to SEQ ID NO:38 of the present invention.

As set forth above, the amylase may comprise an amino acid sequence as shown in SEQ ID NO: 48 (or is a variant thereof). The amylase with SEQ ID NO: 48 is the amylase from Bacillus strain TS-23 having SEQ ID NO:48 of this invention or having SEQ ID NO:2 as disclosed in WO 2009/061380 and variants thereof.

As set forth above, the amylase may comprise an amino acid sequence as shown in SEQ ID NO: 40 (or is a variant thereof). The amylase with SEQ ID NO: 40 (sometimes also referred to as “stainzyme”) is the amylase from Bacillus sp. having SEQ ID NO:40 of this invention or comprising amino acids 1 to 485 of SEQ ID NO:2 as described in WO 00/60060 and variants at least 95% thereto.

In a preferred embodiment of the present invention, the amylase is a hybrid amylase.

As set forth above the amylase may be a hybrid amylase as described in WO 2006/066594. For example, the hybrid amylase may comprise an amino acid sequence as shown in SEQ ID NO: 61 (or is a variant thereof) or may be according to WO 2014/183920 with A and B domains having at least 90% identity to SEQ ID NO:2 of WO 2014/183920 and a C domain having at least 90% identity to SEQ ID NO:6 of WO 2014/183920, wherein the hybrid amylase has amylolytic activity; preferably the hybrid alpha-amylase is at least 95% identical to SEQ ID NO: 23 of WO 2014/183920 and having amylolytic activity. SEQ ID NO: 61 of the invention is 99.4% identical to SEQ ID NO: 23 of WO 2014/183920.

Hybrid amylases may be according to WO 2014/183921 with A and B domains having at least 75% identity to SEQ ID NO: 2, SEQ ID NO: 15, SEQ ID NO: 20, SEQ ID NO: 23, SEQ ID NO: 29, SEQ ID NO: 26, SEQ ID NO: 32, and SEQ ID NO: 39 as disclosed in WO 2014/183921 and a C domain having at least 90% identity to SEQ ID NO: 6 of WO 2014/183921, wherein the hybrid amylase has amylolytic activity; preferably, the hybrid alpha-amylase comprises an amino acid sequence which is at least 95 identical to SEQ ID NO: 6 of WO 2014/183921.

As set forth above the amylase may be a hybrid amylase as disclosed in WO 2021/032881 (herewith incorporated by reference), said amylase comprising an A and B domain originating from the alpha amylase originating from Bacillus sp. A 7-7 (DSM 12368) and a C domain originating from the alpha-amylase from Bacillus cereus; preferably, the A and B domain are at least 75% identical to the amino acid sequence of SEQ ID NO: 42 and a C domain is at least 75% identical to the amino acid sequence of SEQ ID NO: 44—both sequences as disclosed in WO 2021/032881; more preferably, the hybrid amylase is at least 80% identical to SEQ ID NO:54 as disclosed in WO 2021/032881. SEQ ID NO: 54 of WO 2021/032881 corresponds to SEQ ID NO: 29 of the present application.

In accordance with the present invention, the amylase is preferably a variant of the amylase having the sequence as shown in SEQ ID NO: 29, for example, the amylase designated Amy031 or Amy033 (see Examples section).

In a preferred embodiment, the amylase variant has the following substitutions as compared to the amylase comprising an amino acid sequence as shown in SEQ ID NO: 29 (typically using the numbering of SEQ ID NO:30): G4Q, N25H, R176K, G186E, T251E, L405M, and Y482W.

In another preferred embodiment, the amylase variant has the following substitutions as compared to the amylase comprising an amino acid sequence as shown in SEQ ID NO: 29 (typically using the numbering of SEQ ID NO:30): N25H, W116K, R176K, R181T, G186E, N195F, T225A, R320K, and Y482W.

As set forth above, the variants of a parent amylase as referred to herein, i.e. of SEQ ID NO: 29, 35, 38, 40, 42, 48, 50, 53, 59 or 61) shall have amylase activity. Further, it is envisaged that the production of the variant by the host cell of the present invention is increased as compared to the production of the variant by a control cell expressing the first polypeptide and the parent amylase. Thus, the production of the variant amylase shall be increased as compared to the production of the parent amylase.

The amylase that is used in accordance with the present invention is not limited to the above amylases.

In some embodiments of the present invention, the amylase is the amylase from Geobacillus stearothermophilus comprising an amino acid sequence of SEQ ID NO:6 as disclosed in WO 02/10355 or an amylase with optionally having a C-terminal truncation over the wildtype sequence. Suitable variants of SEQ ID NO:6 include those comprising a deletion in positions 179 and/or 181 and/or 182 and/or a substitution in position 193.

In some embodiments of the present invention, the amylase is the amylase from Bacillus sp. 707comprising an amino acid sequence of SEQ ID NO:6 as disclosed in WO 99/19467 and variants at least 95% thereto. Preferred variants of SEQ NO: 6 are those having a substitution, a deletion or an insertion in one or more of the following positions: R181, G182, H183, G184, N195, 1206, E212, E216 and K269.

In some embodiments of the present invention, the amylase is the amylase from Bacillus sp. DSM 12649 having SEQ ID NO:4 as disclosed in WO 00/22103 and variants at least 95% thereto.

In some embodiments of the present invention, the amylase is the amylase from Bacillus megaterium DSM 90 having SEQ ID NO:1 as disclosed in WO 2010/104675 and variants at least 95% thereto.

In some embodiments of the present invention, the amylase is the amylase from Bacillus amyloliquefaciens or variants thereof, preferably selected from amylases according to SEQ ID NO: 3 as described in WO 2016/092009.

In some embodiments of the present invention, the amylase comprises an amino acid sequence as shown in SEQ ID NO:12 as described in WO 2006/002643 or amylase variants thereof comprising the substitutions Y295F and M202LITV within said SEQ ID NO:12.

In some embodiments of the present invention, the amylase comprises an amino acid sequence as shown in SEQ ID NO:6 as described in WO 2011/098531 or amylase variants comprising a substitution at one or more positions selected from the group consisting of 193 [G,A,S,T or M], 195 [F,W,Y,L,I or V], 197 [F,W,Y,L,I or V], 198 [Q or N], 200 [F,W,Y,L,I or V], 203 [F,W,Y,L,I or V], 206 [F,W,Y,N,L,I,V,H,Q,D or E], 210 [F,W,Y,L,I or V], 212 [F,W,Y,L,I or V], 213 [G,A,S,T or M] and 243 [F,W,Y,L,I or V] within said SEQ ID NO:6.

Amylases may have SEQ ID NO:1 as described in WO 2013/001078 or amylase variants comprising an alteration at two or more (several) positions corresponding to positions G304, W140, W189, D134, E260, F262, W284, W347, W439, W469, G476, and G477 within said SEQ ID NO:1.

In some embodiments of the present invention, the amylase comprises an amino acid sequence as shown in SEQ ID NO:2 as described in WO 2013/001087 or amylase variants comprising a deletion of positions 181+182, or 182+183, or 183+184, within said SEQ ID NO:2, optionally comprising one or two or more modifications in any of positions corresponding to W140, W159, W167, Q169, W189, E194, N260, F262, W284, F289, G304, G305, R320, W347, W439, W469, G476 and G477 within said SEQ ID NO:2.

In a preferred embodiment of the present invention, the amylase is a commercially available amylases which include but are not limited to products sold under the trade names Duramyl™, Termamyl™, Fungamyl™, Stainzyme™, Stainzyme Plus™, Natalase™ Liquozyme X and BAN™, Amplify™, Amplify Prime™ (from Novozymes A/S), and Rapidase™, Purastar™, Powerase™, Effectenz™ (M100 from DuPont), Preferenz™ (S1000 DuPont), PrimaGreen™ (ALL; DuPont), Optisize™ (DuPont).

The present invention is not limited to amylase as polypeptide of interest. In some embodiments, the polypeptide of interest (i.e. the second polypeptide) is an enzyme other than an amylase, such as an exoenzyme (other than an amylase). In a particular embodiment, the enzyme is classified as an oxidoreductase (EC 1), a transferase (EC 2), a hydrolase (EC 3), a lyase (EC 4), an isomerase (EC 5), or a ligase (EC 6). In a preferred embodiment, the protein of interest is an enzyme suitable to be used in detergents, feed and food applications.

Most preferably, the enzyme is a hydrolase (EC 3), preferably, a glycosidase (EC 3.2) or a peptidase (EC 3.4). Especially preferred enzymes are enzymes selected from the group consisting of an amylase, a cellulase (EC 3.2.1.4), an endo-1,3-beta-xylanase xylanase (EC 3.2.1.32), an endo-1,4-beta-xylanase (EC 3.2.1.8), a lactase (EC 3.2.1.108), a galactosidase (EC 3.2.1.23 and EC 3.2.1.24), a mannanase (EC 3.2.1.24 and EC 3.2.1.25), a lipase (EC 3.1.1.3), a phytase (EC 3.1.3.8), a nuclease (EC 3.1.11 to EC 3.1.31), and a protease (EC 3.4); in particular an enzyme selected from the group consisting of amylase, protease, lipase, mannanase, phytase, xylanase, phosphatase, 8-galactosidase, lactase, glucoamylase, nuclease, and cellulase, preferably, amylase, mannanase, xylanase or protease, preferably, a protease.

In some embodiments, the second polypeptide is a xylanase,

In some embodiments, the second polypeptide is a mannanase.

### Signal Peptide-Secretion Signal for the Polypeptide of Interest

The polypeptide of interest, such as the amylase polypeptide, as referred to herein is secreted, i.e. it is secreted by the Bacillus licheniformis host cell of the present invention. Thus, the polypeptide of interest, preferably an amylase, typically comprises—at the N-terminus—a secretion signal, i.e. a signal peptide which allows for secretion of the polypeptide from the host cell into the fermentation broth. Typically, the signal peptide is Sec-secretion-pathway-specific signal peptide. Thus, the polypeptide of interest is secreted via the Sec pathway. Typically, the signal peptide is present at the N-terminus of the polypeptide of interest.

The terms “signal peptide”, “secretion sequence”, “secretion signal peptide” are used interchangeably herein.

Signal peptides are well-known in the art and can be found, in general, at the N-terminus of secreted Bacillus proteins, such as the of AmyB, AmyE, AmyL AmyM, AmyQ, AmyS, AprE, AprH, AspB, BglC, BglS, Bpr, CelA, CelA, Csn, Epr, ForD, GGT, LacZ, LipA, LytB, LytD, Pel, PhoD, PhrK, Vpr, YbdN, YckD, YddT, YfhK, YfjS, YhfM, YjfA, YkwD, YncM, YnfF, YobB, YvcE, or YvfO polypeptide.

It is also within the scope of the present invention that signal peptide sequences can be engineered by replacing amino acids or creating chimeric sequences to optimize secretion of the polypeptide of interest (EP26890151B1).

Signal peptides may also be selected from signal peptides that comprise both Sec-secretion-pathway and TAT-secretion-pathway sequence elements (Freudl, R. Signal peptides for recombinant protein secretion in bacterial expression systems. Microb Cell Fact 17, 52 (2018)) such as but not limited to the signal peptides of the WprA, WapA, SpoIIP or YwbN polypeptide.

Exemplary signal peptides as shown in the following Table A. In a preferred embodiment, the polypeptide comprises, at the N-terminus, a signal peptide as shown in Table A. Accordingly, the signal peptide, preferably, comprises or consists of an amino acid sequence selected from SEQ ID NO: 67 to 109.

TABLE A
Exemplary signal peptides from various Bacillus polypeptides

| SEQ ID | | | |
|---|---|---|---|
| No | Species | Origin | Sequence |
| 67 | Bacillus cereus | signal peptide | MKNQFQYCCIVILSVVMLFVSLLIPQASSA |
| | | AmyB | |
| 68 | Bacillus subtilis | signal peptide | MFAKRFKTSLLPLFAGFLLLFHLVLAG |
| | | AmyE | |
| 69* | Bacillus | signal peptide | MKQQKRLYARLLTLLFALIFLLPHSAAAA |
| | licheniformis | AmyL | |
| 70 | Bacillus | signal peptide | MKKKTLSLFVGLMLLIGLLFSGSLPYNPNAAEA |
| | stearothermophilus | AmyM | |
| 71 | Bacillus | signal peptide | MIQKRKRTVSFRLVLMCTLLFVSLPITKTSA |
| | amyloliquefaciens | AmyQ | |
| 72 | Bacillus | signal peptide | MLTFHRIIRKGWMFLLAFLLTALLFCPTGQPAKA |
| | stearothermophilus | AmyS | |
| 73* | Bacillus subtilis | signal peptide | MRSKKLWISLLFALTLIFTMAFSNMSAQA |
| | | AprE | |
| 74 | Bacillus | signal peptide | MMRKKSFWLGMLTAFMLVFTMASIASA |
| | licheniformis | AprE | |
| 75 | Bacillus pumilus | signal peptide | MKKKNVMTSVLLAVPLLFSAGFGGSMAQA |
| | | AprE | |
| 76 | Bacillus clausii | signal peptide | MKKPLGKIVASTALLISVAFSSSIASA |
| | | AprH | |
| 77 | Bacillus subtilis | signal peptide | MKLAKRVSALTPSTTLAITAKA |
| | | AspB | |
| 78 | Bacillus subtilis | signal peptide | MKRSISIFITCLLITLLTMGGMIASPASA |
| | | BglC | |
| 79 | Bacillus subtilis | signal peptide | MPYLKRVLLLLVTGLFMSLFAVTATASA |
| | | BglS | |
| 80 | Bacillus subtilis | signal peptide | MRKKTKNRLISSVLSTVVISSLLFPGAAGA |
| | | Bpr | |
| 81 | Streptomyces | signal peptide | MGFGSAPIALCPLRTRRNALKRLLALLATGVSIVGLT |
| | lividans | CelA | ALAGPPAQA |
| 82 | Bacillus | signal peptide | MMAEKVFSKNKIIGGKRMSYMKRSISVFIACFMVAV |
| | licheniformis | CelA | LGISGIIAPKASA |
| 83 | Bacillus subtilis | signal peptide | MKISMQKADFWKKAAISLLVFTMFFTLMMSETVFA |
| | | Csn | |
| 84 | Bacillus subtilis | signal peptide | MKNMSCKLVVSVTLFFSFLTIGPLAHA |
| | | Epr | |
| 85 | Bacillus | signal peptide | MKNHLYEKKKRKPLTRTIKATLAVLTMSIALVGGATV |
| | licheniformis | ForD | PSLA |
| 86 | Bacillus | signal peptide | MRRLAFLVVAFCLAVGCFFSPVSKA |
| | licheniformis | GGT | |
| 87 | Bifidobacterium | signal peptide | MAVRRLGGRIVAFAATVALSIPLGLLTNSAWA |
| | bifidum | LacZ | |
| 88 | Bacillus subtilis | signal peptide | MKFVKRRIIALVTILMLSVTSLFALQPSAKA |
| | | LipA | |
| 89 | Bacillus subtilis | signal peptide | MKSCKQLIVCSLAAILLLIPSVSFA |
| | | LytB | |
| 90 | Bacillus subtilis | signal peptide | MKKRLIAPMLLSAASLAFFAMSGSAQA |
| | | LytD | |
| 91 | Bacillus subtilis | signal peptide | MKKVMLATALFLGLTPAGANA |
| | | Pel | |
| 92 | Bacillus subtilis | signal peptide | MAYDSRFDEWVQKLKEESFQNNTFDRRKFIQGAGKI |
| | | PhoD | AGLSLGLTIAQSVGA |
| 93 | Bacillus subtilis | signal peptide | MKKLVLCVSILAVILSGVA |
| | | PhrK | |
| 94 | Bacillus subtilis | signal peptide | MKKGIIRFLLVSFVLFFALSTGITGVQAAPA |
| | | Vpr | |
| 95 | Bacillus subtilis | signal peptide | MKKRKRRNFK RFIAAFLVLALMISLVPADVLA |
| | | WapA | |
| 96 | Bacillus subtilis | signal peptide | MVKKWLIQFAVMLSVLSTFTYSASA |
| | | YbdN | |
| 97 | Bacillus subtilis | signal peptide | MKRITINIITMFIAAAVISLTGTAEA |
| | | YckD | |
| 98 | Bacillus subtilis | signal peptide | MRKKRVITCVMAASLTLGSLLPAGYASA |
| | | YddT | |
| 99 | Bacillus subtilis | signal peptide | MKKKQVMLALTAAAGLGLTALHSAPAAKA |
| | | YfhK | |
| 100 | Bacillus subtilis | signal peptide | MKWMCSICCAAVLLAGGAAQA |
| | | YfjS | |
| 101 | Bacillus subtilis | signal peptide | MKKIVAAIVVIGLVFIAFFYLYSRSGDVYQSVDA |
| | | YhfM | |
| 102 | Bacillus subtilis | signal peptide | MKRLFMKASLVLFAVVFVFAVKGAPAKA |
| | | YjfA | |
| 103 | Bacillus subtilis | signal peptide | MKKAFILSAAAAVGLFTFGGVQQASA |
| | | YkwD | |
| 104 | Bacillus subtilis | signal peptide | MAKPLSKGGILVKKVLIAGAVGTAVLFGTLSSGIPGL |
| | | YncM | PAADA |
| 105 | Bacillus subtilis | signal peptide | MIPRIKKTICVLLVCFTMLSVMLGPGATEVLA |
| | | YnfF | |
| 106 | Bacillus subtilis | signal peptide | MKIRKILLSSALSFGMLISAVPALA |
| | | YobB | |
| 107 | Bacillus subtilis | signal peptide | MRKSLITLGLASVIGTSSFLIPFTSKTASA |
| | | YvcE | |
| 108 | Bacillus | signal peptide | MKNVLAVFVVLIFVLGAFGTSGPAEA |
| | licheniformis | YvfO | |
| 109 | Bacillus subtilis | signal peptide | MSDEQKKPEQIHRRDILKWGAMAGAAVAIGASGLGG |
| | | YwbN | LAPLVASA |

*were used in the Examples section

In one embodiment, the signal peptide comprises or consists of an amino acid sequence as shown in SEQ ID NO: 69, 73 or 107.

As set forth above, preferably the polypeptide of carries functional signal peptide.

Whether a peptide acts as secretion signal peptide, or not, can be assessed by a bioinformatics approach using SignalP signal peptide prediction tool (Almagro Armenteros J J, Nielsen H.; SignalP 5.0 improves signal peptide predictions using deep neural networks. Nat Biotechnol. 2019 April; 37(4):420-423); or by measuring the secretion ability for a given polypeptide when fused to a potential secretion signal peptide as previously shown (Brockmeier U, Eggert T. Systematic screening of all signal peptides from Bacillus subtilis: a powerful strategy in optimizing heterologous protein secretion in Gram-positive bacteria. J Mol Biol. 2006 Sep. 22; 362(3):393-402).

### Expression Constructs

The host cell of the present invention, preferably, comprises a polynucleotide encoding the first polypeptide and a polynucleotide encoding the second polypeptide. Thus, the host cell preferably comprises
a) a first expression cassette for said first polypeptide, said first expression cassette comprising a promoter, operably linked to a first polynucleotide encoding said first polypeptide, and optionally a terminator, and
b) a second expression cassette expressing for said second polypeptide, said second expression cassette comprising a promoter, operably linked to a second polynucleotide encoding said second polypeptide, and optionally a terminator.

Preferably, both polynucleotides, i.e. the polynucleotide encoding the first polypeptide and a polynucleotide encoding the second polypeptide the polynucleotide encoding at least one polypeptide of interest are heterologous to the host cell. The term “heterologous” (or exogenous or foreign or recombinant or non-native), typically, refers to a polynucleotide that is not native to the host cell. In some embodiments, a “heterologous” polynucleotide is an additional copy of a gene that naturally occurs in in the host. The term “heterologous” (or exogenous or foreign or recombinant or non-native) polypeptide or protein as used throughout the specification is defined herein as a polypeptide or protein that is not native to the host cell.

In a preferred embodiment, the first and the second polynucleotide, and thus, the first and the second expression cassette are present on a plasmid. The term “plasmid” refers to an extrachromosomal circular DNA, i.e. a vector that is autonomously replicating in the host cell. Thus, a plasmid is understood as extrachromosomal vector.

In another preferred embodiment, the first and the second polynucleotide, and thus, the first and the second expression cassette are stably integrated into the bacterial chromosome.

### Promoter

The first and the second polynucleotide shall be operably linked to a promoter.

The term “operably linked” as used herein refers to a functional linkage between the promoter sequence and the polynucleotide encoding a polypeptide of interest, such that the promoter sequence is able to initiate transcription of the polynucleotide encoding a polypeptide of interest (herein also referred to as gene of interest).

A “promoter” or “promoter sequence” is a nucleotide sequence located upstream of a gene on the same strand as the gene that enables that gene's transcription. A promoter is followed by the transcription start site of the gene. A promoter is recognized by RNA polymerase (together with any required transcription factors), which initiates transcription. A functional fragment or functional variant of a promoter is a nucleotide sequence which is recognizable by RNA polymerase, and capable of initiating transcription.

An “active promoter fragment”, “active promoter variant”, “functional promoter fragment” or “functional promoter variant” describes a fragment or variant of the nucleotide sequences of a promoter, which still has promoter activity.

A promoter can be an “inducer-dependent promoter” or an “inducer-independent promoter” comprising constitutive promoters or promoters which are under the control of other cellular regulating factors.

The person skilled in the art is capable to select suitable promoters for expressing the first and the second polynucleotide. For example, the polynucleotide encoding the first polypeptide of interest is, preferably, operably linked to an “inducer-dependent promoter” or an “inducer-independent promoter”. Further, the polynucleotide encoding the second polypeptide of interest is preferably, operably linked to an “inducer-independent promoter”, such as a constitutive promoter.

An “inducer dependent promoter” is understood herein as a promoter that is increased in its activity to enable transcription of the gene to which the promoter is operably linked upon addition of an “inducer molecule” to the fermentation medium. Thus, for an inducer-dependent promoter, the presence of the inducer molecule triggers via signal transduction an increase in expression of the gene operably linked to the promoter. The gene expression prior activation by the presence of the inducer molecule does not need to be absent, but can also be present at a low level of basal gene expression that is increased after addition of the inducer molecule. The “inducer molecule” is a molecule, the presence of which in the fermentation medium is capable of affecting an increase in expression of a gene by increasing the activity of an inducer-dependent promoter operably linked to the gene. Preferably, the inducer molecule is a carbohydrate or an analog thereof. In one embodiment, the inducer molecule is a secondary carbon source of the Bacillus cell. In the presence of a mixture of carbohydrates, cells selectively take up the carbon source that provide them with the most energy and growth advantage (primary carbon source). Simultaneously, they repress the various functions involved in the catabolism and uptake of the less preferred carbon sources (secondary carbon source). Typically, a primary carbon source for Bacillus is glucose and various other sugars and sugar derivates being used by Bacillus as secondary carbon sources. Secondary carbon sources include e.g. mannose or lactose without being restricted to these.

Examples of inducer dependent promoters are given in the table below by reference to the respective operon:

| Operon | Regulator a) | Type b) | Inducer | Organism |
|---|---|---|---|---|
| sacPA | SacT | AT | sucrose | B. subtilis |
| sacB | SacY | AT | sucrose | B. subtilis |
| bgl PH | LicT | AT | β-glucosides | B. subtilis |
| licBCAH | LicR | A | oligo-β- | B. subtilis |
| | | | glucosides | |
| levDEFG sacL | LevR | A | fructose | B. subtilis |
| mtlAD | MtlR | A | mannitol | B. subtilis |
| manPA-yjdF | ManR | A | mannose | B. subtilis |
| manR | ManR | A | mannose | B. subtilis |
| bglFB bglG | BglG | AT | β-glucosides | E. coli |
| lacTEGF | LacT | AT | lactose | L. casei |
| lacZYA | lacl | R | Allolactose; | E. coli |
| | | | IPTG (Isopropyl | |
| | | | β-D-1-thio- | |
| | | | galactopy- | |
| | | | ranoside) | |
| araBAD | araC | AR | L-arabinose | E. coli |
| xylAB | XylR | R | Xylose | B. subtilis |

a) transcriptional regulator protein
b) A: activator
AT: antiterminator
R: repressor
AR: activator/repressor

In contrast thereto, the activity of promoters that do not depend on the presence of an PG-3T inducer molecule (herein called ‘inducer-independent promoters’) are either constitutively active or can be increased regardless of the presence of an inducer molecule that is added to the fermentation medium.

Constitutive promoters are independent of other cellular regulating factors and transcription initiation is dependent on sigma factor A (sigA). The sigA-dependent promoters comprise the sigma factor A specific recognition sites ‘-35’-region and ‘-10’-region.

Preferably, the ‘inducer-independent promoter’ sequence is selected from the group consisting of constitutive promoters not limited to promoters Pveg, PlepA, PserA, PymdA, Pfba and derivatives thereof with different strength of gene expression (Guiziou et al, (2016): Nucleic Acids Res. 44(15), 7495-7508), the aprE promoter of Subtilisin encoding aprE gene of Bacilli, the bacteriophage SPO1 promoters P4, P5, P15 (WO15118126), the cryIIIA promoter from Bacillus thuringiensis (WO9425612), the amyQ promoter from Bacillus amyloliquefaciens, the amyL promoter and promoter variants from Bacillus licheniformis (U.S. Pat. No. 5,698,415) and combinations thereof, or active fragments or variants thereof, preferably an aprE promoter sequence.

An “aprE promoter” or “aprE promoter sequence” is the nucleotide sequence (or parts or variants thereof) located upstream of an aprE gene, i.e., a gene coding for a Bacillus Subtilisin Carlsberg protease, on the same strand as the aprE gene that enables that aprE gene's transcription.

In one embodiment of the present invention, the promoter is the promoter of an aprE gene, such as a promoter of the Bacillus licheniformis aprE gene (which was used in the Examples section). For example, the promoter comprises a nucleic acid sequence as shown in SEQ ID NO: 3 or a nucleic acid sequence being at least 80%, 85%, 90%, 93%, 95%, 98% or 99% identical to SEQ ID NO: 3.

For the co-expression of prsA genes in the Bacillus host, the native 5′ prsA gene regulatory region of the prsA gene can be used (WO9419471, WO2021146411) as well as heterologous promoters (WO2020156903). Inducible promoters such as the IPTG inducible promoter Pspac and the xylose-inducible promoter PxyIA have been used for titration of the PrsA expression level within the cell (Chen J et al. Biotechnol Lett. 2015 April; 37(4):899-906. doi: 10.1007/s10529-014-1755-3. Epub 2014 Dec. 17. PMID: 25515799.).

The term “transcription start site” or “transcriptional start site” shall be understood as the location where the transcription starts at the 5′ end of a gene sequence. In prokaryotes the first nucleotide, referred to as +1 is in general an adenosine (A) or guanosine (G) nucleotide. In this context, the terms “sites” and “signal” can be used interchangeably herein.

The term “expression” or “gene expression” means the transcription of a specific gene or specific genes or specific nucleic acid construct. The term “expression” or “gene expression” in particular means the transcription of a gene or genes or genetic construct into structural RNA (e.g., rRNA, tRNA) or mRNA with or without subsequent translation of the latter into a protein. The process includes transcription of DNA and processing of the resulting mRNA product.

Further optionally the promoter comprises a 5′UTR. This is a transcribed but not translated region downstream of the −1 promoter position. Such untranslated region for example should contain a ribosome binding site to facilitate translation in those cases where the target gene codes for a peptide or polypeptide.

With respect to the 5′UTR the invention in particular teaches to combine the promoter of the present invention with a 5′UTR comprising one or more stabilizing elements. This way the mRNAs synthesized from the promoter region may be processed to generate mRNA transcript with a stabilizer sequence at the 5′ end of the transcript. Preferably such a stabilizer sequence at the 5′end of the mRNA transcripts increases their half-life as described by Hue et al, 1995, Journal of Bacteriology 177: 3465-3471. Suitable mRNA stabilizing elements are those described in
WO08148575, preferably SEQ ID NO. 1 to 5 of WO08140615, or fragments of these sequences which maintain the mRNA stabilizing function, and in
WO08140615, preferably Bacillus thuringiensis CryIIIA mRNA stabilising sequence or bacteriophage SP82 mRNA stabilising sequence, more preferably a mRNA stabilising sequence according to SEQ ID NO. 4 or 5 of WO08140615, more preferably a modified mRNA stabilising sequence according to SEQ ID NO. 6 of WO08140615, or fragments of these sequences which maintain the mRNA stabilizing function.

Preferred mRNA stabilizing elements are selected from the group consisting of aprE grpE, cotG, SP82, RSBgsiB, CryIIIA mRNA stabilizing elements, or according to fragments of these sequences which maintain the mRNA stabilizing function. A preferred mRNA stabilizing element is the grpEmRNA stabilizing element (corresponding to SEQ ID NO. 2 of WO08148575).

The 5′UTR also preferably comprises a modified rib leader sequence located downstream of the promoter and upstream of a ribosome binding site (RBS). In the context of the present invention a rib leader is herewith defined as the leader sequence upstream of the riboflavin biosynthetic genes (rib operon) in a Bacillus cell, more preferably in a Bacillus subtilis cell. In Bacillus subtilis, the rib operon, comprising the genes involved in riboflavin biosynthesis, include ribG (ribD), ribB (ribE), ribA, and ribH genes. Transcription of the riboflavin operon from the rib promoter (Prib) in B. subtilis is controlled by a riboswitch involving an untranslated regulatory leader region (the rib leader) of almost 300 nucleotides located in the 5′-region of the rib operon between the transcription start and the translation start codon of the first gene in the operon, ribG. Suitable rib leader sequences are described in WO2015/1181296, in particular pages 23-25, incorporated herein by reference.

The definitions and explanations provided herein above, apply mutatis mutandis to the following.

### Method for Producing a Polypeptide of Interest

The present invention further relates to a method for producing a polypeptide of interest, such as a polypeptide having amylase activity. Preferably, said method comprises the following step:
a) providing the Bacillus licheniformis host cell of the present invention, and
b) cultivating the Bacillus licheniformis host cell under conditions that allow for expressing said polypeptide having amylase activity, and optionally,
c) obtaining or purifying said polypeptide of interest, such as the polypeptide having amylase activity.

The term “cultivating” as used herein refers to keeping alive and/or propagating the modified host cell comprised in a culture at least for a predetermined time. The term encompasses phases of exponential cell growth at the beginning of growth after inoculation as well as phases of stationary growth. The cultivation conditions shall allow for the expression, i.e. the production, of the polypeptide of interest. Such conditions can be chosen by the skilled person without further ado. Exemplary conditions for the cultivation of the modified host cell are described in WO2020169564 A1 or in Example 1 or in Example 2 in the Examples section. In an embodiment of the method of the present invention, the cultivation in step b) is carried out as fed batch cultivation.

The method of the present invention, if applied, allows for increasing the production, of the at least one polypeptide of interest. Preferably, production is increased as compared to the expression in an unmodified control cell, i.e. a Bacillus licheniformis control cell which does not express the first polypeptide as referred to herein. In a preferred embodiment, the production of the polypeptide of interest is increased by at least 20%, such as by at least 50%, in particular by at least 100% or at least 200% as compared to the expression in the control cell. For example, the production of the polypeptide of interest may be increased by 20% to 300%, such as by 100% to 300% as compared to the control cell. The expression can be measured by determining the amount of the polypeptide in the host cell and/or in the cultivation medium. Further, the production may be assessed by determining the enzymatic activity. For example, an enzyme assay may be used to determine the activity of the polypeptide of interest.

The polypeptide of interest may be obtained or purified using methods known in the art. For example, the polypeptide may be obtained from the cultivation medium by methods, such centrifugation, filtration, extraction, spray drying, or precipitation.

The polypeptide of interest may be purified by any method deemed appropriate such as but ion exchange chromatography, electrophoretic procedures, SDS-PAGE, or extraction.

Method for Producing the Bacillus licheniformis Host Cell of the Present Invention

The present invention further relates to a method of producing the Bacillus licheniformis host cell of the present invention, comprising
a) providing a Bacillus licheniformis host cell, and
b) introducing into the host cell provided in step a)
   b1) a first polynucleotide encoding a first polypeptide having peptidyl-prolyl cis-trans isomerase (EC 5.2.1.8) activity, said first polypeptide comprising an amino acid sequence as shown in SEQ ID NO: 1, or an amino acid sequence being a least 81% identical to SEQ ID NO: 1, and
   b2) a second polynucleotide encoding a second polypeptide having amylase activity.

The introduction in step b) can be done by any method deemed appropriate such as by transformation, e.g. with one or more plasmids comprising the first and/or second polynucleotide. The plasmid preferably comprises a selectable marker gene.

The present invention further relates to the use of
i) a first polypeptide having peptidyl-prolyl cis-trans isomerase (EC 5.2.1.8) activity, said first polypeptide comprising an amino acid sequence as shown in SEQ ID NO: 1, or an amino acid sequence being a least 81% identical to SEQ ID NO: 1, and/or
ii) a polynucleotide encoding said first polypeptide

for increasing the production of a second polypeptide having alpha amylase activity in a Bacillus licheniformis host cell.

Finally, the present invention relates to the use of the Bacillus licheniformis host cell of the present invention for producing a polypeptide having alpha amylase activity.

Throughout this application, various publications are referenced. The disclosures of all of these publications and those references cited within those publications in their entireties are hereby incorporated by reference into this application in order to more fully describe the state of the art to which this invention pertains.

### EXAMPLES

### Materials and Methods

The following examples only serve to illustrate the invention. The numerous possible variations that are obvious to a person skilled in the art also fall within the scope of the invention.

Unless otherwise stated the following experiments have been performed by applying standard equipment, methods, chemicals, and biochemicals as used in genetic engineering, molecular biology and fermentative production of chemical compounds by cultivation of microorganisms. See also Sambrook et al. (Sambrook, J. and Russell, D. W. Molecular cloning. A laboratory manual, 3^{rd} ed, Cold Spring Harbor Laboratory Press, Cold Spring Harbor, NY. 2001).

Electrocompetent Bacillus licheniformis cells and electroporation Transformation of DNA into Bacillus licheniformis (U.S. Pat. No. 5,352,604) is performed via electroporation. Preparation of electrocompetent Bacillus licheniformis cells and transformation of DNA is performed as essentially described by Brigidi et al (Brigidi, P., Mateuzzi, D. (1991). Biotechnol. Techniques 5, 5) with the following modification: Upon transformation of DNA, cells are recovered in 1 ml LBSPG buffer and incubated for 60 min at 37° C. (Vehmaanpers J., 1989, FEMS Microbio. Lett., 61: 165-170) following plating on selective LB-agar plates.

In order to overcome the Bacillus licheniformis specific restriction modification system of Bacillus licheniformis strains, plasmid DNA is isolated from Ec #098 cells or B. subtilis Bs #056 cells as described below.

Plasmid Isolation Plasmid DNA was isolated from Bacillus and E. coli cells by standard molecular biology methods described in (Sambrook, J. and Russell, D. W. Molecular cloning. A laboratory manual, 3rd ed, Cold Spring Harbor Laboratory Press, Cold Spring Harbor, NY. 2001) or the alkaline lysis method (Birnboim, H. C., Doly, J. (1979). Nucleic Acids Res 7(6): 1513-1523). Bacillus cells were in comparison to E. coli treated with 10 mg/ml lysozyme for 30 min at 37° C. prior to cell lysis.

### Plasmids

Plasmid p689-T2A-lac

The E. coli plasmid p689-T2A-lac comprises the lacZ-alpha gene flanked by Bpil restriction sites, again flanked 5′ by the T1 terminator of the E. coli rrnB gene and 3′ by the TO lambda terminator and was ordered as gene synthesis construct (SEQ ID NO:6).

Plasmid pEC194RS—Bacillus temperature sensitive deletion plasmid (WO2022018260) is used for the cloning of gene deletion and gene integration constructs.

Plasmid pBIL013: Bacillus subtilis Integration Destination Plasmid

The plasmid pBIL013 is a gene integration plasmid with homology regions of the 5′ and 3′ regions of the aprE gene of B. subtilis and a Type-II clone cassette with a chloramphenicol resistance gene as selectable marker. The plasmid backbone of plasmid BIL009 (WO2019016051) was PCR amplified with oligonucleotides SEQ ID NO:18 and SEQ ID NO:19. The 5′ homology and 3′ homology regions of the aprE gene were PCR-amplified with oligonucleotides SEQ ID NO:20, SEQ ID NO:21, SEQ ID NO:22 and SEQ ID NO:23 respectively. The type-type clone cassette was provided a gene synthesis construct (BioCat GmbH, Heidelberg) SEQ ID NO:24. The individual genetic elements were assembled via Type-II cloning and BsaI restriction endonuclease as described (Radeck et al., 2017; Sci. Rep. 7: 14134) and the reaction mixture subsequently transformed into E. coli DH10B cells (Life technologies). Transformants were spread and incubated overnight at 37° C. on LB-agar plates containing 25 μg/ml kanamycin. Plasmid DNA was isolated from individual clones and analyzed for correctness by restriction digest. The resulting plasmid pBIL013 was sequence-verified.

Gene integration plasmids Bacillus licheniformis—pInt
pInt020—Cat::PaprE-prsA_Bpu Integration Plasmid:

The Cat::PaprE-prsA_Bpu integration plasmid for integration of the Bacillus pumilus prsA gene (prsA_Bpu; SEQ ID NO:2) under control of the Bacillus licheniformis aprE gene promoter (SEQ ID NO:3) into the chloramphenicol-acetyltransferase (Cat) locus was constructed by a two-step cloning strategy. First, the prsA_Bpu expression cassette comprising the Bacillus licheniformis aprE gene promoter and the Bacillus pumilus prsA gene were ordered as gene synthesis fragments with flanking Bpil restriction endonuclease sites (SEQ ID NO:4 and SEQ ID NO:5 respectively) and subcloned into p689-T2A-lac in a type-II-assembly reaction with restriction endonuclease Bpil as described (Radeck et al., 2017; Sci. Rep. 7: 14134) and the reaction mixture subsequently transformed into E. coli DH10B cells (Life technologies). Transformants were spread and incubated overnight at 37° C. on LB-agar plates containing 25 μg/ml kanamycin. Plasmid DNA was isolated from individual clones and analyzed for correctness by restriction digest. The resulting plasmid p689-PaprE-prsA_Bpu was sequence-verified.

The plasmid for exchange of the chloramphenicol acetyltransferase (Cat) gene locus (SEQ ID NO:7) by the prsA_Bpu expression cassette was constructed in a second type-II-assembly reaction with restriction endonuclease BsaI with plasmids pEC194RS and p689-PaprE-prsA_Bpu, and 5′ and 3′ homology regions of the Cat gene locus (SEQ ID NO:8 and SEQ ID NO:9) provided as gene synthesis constructs flanked by BsaI restriction sites compatible with pEC194RS and p689-PaprE-prsA_Bpu to allow for directed cloning. The type-II-assembly with restriction endonuclease BsaI was performed as described and the reaction mixture subsequently transformed into E. coli Ec #098. Transformants were spread and incubated overnight at 37° C. on LB-agar plates containing 100 μg/ml ampicillin and 30 μg/ml chloramphenicol. Plasmid DNA was isolated from individual clones and analyzed for correctness by sequencing. The resulting sequence-verified plasmid was named pInt020_prsA_Bpu.

Gene integration plasmids for prsA genes from other species were constructed as described for plasmid pInt20 and are listed in Table 1.

TABLE 1

| Plasmid Name | Promoter | prsA gene | Species | Target locus |
|---|---|---|---|---|
| plnt20_prsA_Bpu | PaprE | SEQ ID NO: 2 | B. pumilus | Cat gene |
| | (SEQ ID NO: 3) | | | (SEQ ID NO: 7) |
| plnt21_prsA_Bli | PaprE | SEQ ID NO: 13 | B. licheniformis | Cat gene |
| | (SEQ ID NO: 3) | | | (SEQ ID NO: 7) |
| plnt22_prsA_Ble | PaprE | SEQ ID NO: 15 | B. lentus | Cat gene |
| | (SEQ ID NO: 3) | | | (SEQ ID NO: 7) |
| plnt23_prsA_Gst | PaprE | SEQ ID NO: 17 | G. steaother- | Cat gene |
| | (SEQ ID NO: 3) | | mophilus | (SEQ ID NO: 7) |

Plasmid pUK57: Type-II-Assembly Destination Bacillus Plasmid

Plasmid pUK57 (described in WO2022018260) is a derivative of plasmid pUB110 comprising a type-II cloning cassette for assembly of gene expression vectors.

### Amylase Expression Plasmids

The amylase expression plasmids are each composed of 3-4 genetic elements—the plasmid backbone of pUK57, the promoter fragment of the aprE gene from Bacillus licheniformis (SEQ ID NO:3), or the signal peptide-amylase gene fragment or the signal peptide fragment and the amylase gene fragment respectively. The pUK57 vector, the promoter fragment (SEQ ID NO:4), the signal peptide-amylase gene fragment or the signal peptide gene fragment and the amylase gene fragment each comprising compatible type-II restriction endonuclease Bpil sites (see Table 2) were assembled in an in vitro type-II-assembly reaction with restriction endonuclease Bpil as described (Radeck et al., 2017; Sci. Rep. 7: 14134) and the reaction mixture subsequently transformed into B. subtilis Bs #056 cells made competent according to the method of Spizizen (Anagnostopoulos, C. and Spizizen, J. (1961). J. Bacteriol. 81, 741-746) following plating on LB-agar plates with 20 μg/ml Kanamycin. Correct clones of final amylase plasmids were analyzed by restriction enzyme digest and sequencing. Table 2 summarizes the Amylase expression plasmids

TABLE 2
Amylase expression plasmids

| Plasmid | Signalpeptide | Amylase | Amylase |
|---|---|---|---|
| Name | fragment | fragment | mature protein |
| pAMY029 | aprE | SEQ ID NO: 28 | SEQ ID NO: 29 |
| | (SEQ ID NO: 26) | | |
| pAMY031 | aprE | Gene variant | Amylase variant |
| | (SEQ ID NO: 26) | of | of |
| | | SEQ ID NO: 28 | SEQ ID NO: 29 |
| pAMY033 | aprE | Gene variant | Amylase variant |
| | (SEQ ID NO: 26) | of | of |
| | | SEQ ID NO: 28 | SEQ ID NO: 29 |
| pAMY035 | amyL | SEQ ID NO: 34 | SEQ ID NO: 35 |
| | (SEQ ID NO: 27 | | |
| pAMY038 | nt 11-103 | nt 104-1561 | SEQ ID NO: 38 |
| | SEQ ID NO: 36 | SEQ ID NO: 36 | |
| pAMY040 | aprE | SEQ ID NO: 39 | SEQ ID NO: 40 |
| | (SEQ ID NO: 26) | | |
| pAMY042 | nt 11-91 | nt 92-1552 | SEQ ID NO: 42 |
| | SEQ ID NO: 41 | SEQ ID NO: 41 | |
| pAMY045 | aprE | SEQ ID NO: 44 | SEQ ID NO: 45 |
| | (SEQ ID NO: 26) | | |
| pAMY048 | aprE | SEQ ID NO: 47 | SEQ ID NO: 48 |
| | (SEQ ID NO: 26) | | |
| pAMY050 | Nt 11-94 | Nt 95-1549 | SEQ ID NO: 50 |
| | SEQ ID NO: 49 | SEQ ID NO: 49 | |
| pAMY053 | aprE | SEQ ID NO: 52 | SEQ ID NO: 53 |
| | (SEQ ID NO: 26) | | |
| pAMY057 | aprE | SEQ ID NO: 55 | SEQ ID NO: 57 |
| | (SEQ ID NO: 26) | | |
| pAMY059 | aprE | SEQ ID NO: 58 | SEQ ID NO: 59 |
| | (SEQ ID NO: 26) | | |
| pAMY061 | aprE | SEQ ID NO: 60 | SEQ ID NO: 61 |
| | (SEQ ID NO: 26) | | |

Plasmid pAmy031

| | |
|---|---|
| Amylase | SEQ ID NO: 29 with substitutions G4Q + N25H + |
| variant | R176K + G186E + T251E + L405M + Y482W (*) |

Plasmid pAmy033

| | |
|---|---|
| Amylase | SEQ ID NO: 29 with substitutions N25H + W116K + |
| variant | R176K + R181T + G186E + N195F + T225A + |
| | R320K + Y482W (*) |

(*) Amylase variant of SEQ ID NO: 29 with the mutations given in the numbering of SEQ ID NO: 30

### Strains

E. coli Strain Ec #098

E. coli strain Ec #098 is an E. coli INV110 strain (Life technologies) carrying the DNA-methyltransferase encoding expression plasmid pMDS003 WO2019016051.

B. subtilis Strain Bs #056

The prototrophic Bacillus subtilis strain KO-7S (BGSCID: 1S145; Zeigler D. R.) was made competent according to the method of Spizizen (Anagnostopoulos, C. and Spizizen, J. (1961). J. Bacteriol. 81, 741-746.) and transformed with the linearized DNA-methyltransferase expression plasmid pMIS012 for integration of the DNA-methyltransferase into the amyE gene as described for the generation of B. subtilis Bs #053 in WO2019/016051. Cells were spread and incubated overnight at 37° C. on LB-agar plates containing 10 μg/ml chloramphenicol. Grown colonies were picked and stroke on both LB-agar plates containing 10 μg/ml chloramphenicol and LB-agar plates containing 10 μg/ml chloramphenicol and 0.5% soluble starch (Sigma) following incubation overnight at 37° C. The starch plates were covered with iodine containing Lugols solution and positive integration clones identified with negative amylase activity. Genomic DNA of positive clones was isolated by standard phenol/chloroform extraction methods after 30 min treatment with lysozyme (10 mg/ml) at 3° C., following analysis of correct integration of the MTase expression cassette by PCR. The resulting B. subtilis strain is named Bs #056.

B. subtilis Strains

The prototrophic Bacillus subtilis strain KO-7S (BGSCID: 1S145; Zeigler D. R.) was used for integration of the various prsA genes. The Bacillus subtilis strain KO-7S and its derivatives was made competent as described for B. subtilis Bs #056.

B. subtilis Strains with Integrated prsA Expression Cassettes

The prsA genes as listed in Table 3 were cloned into the pBIL013 plasmid together the promoter of the B. licheniformis secA gene (WO2019016051, SEQ ID NO:25) via Type-II cloning with Bpil restriction endonuclease as described. The assembled plasmids were linearized with restriction endonuclease BsmbI and the reaction mixture subsequently transformed into competent Bacillus subtilis strain KO-7S as described for Bs #056. Correct gene integrations were screened for growth on selective chloramphenicol LB-agar plates and kanamycin sensitivity. Finally, the correct integration of the prsA gene expression cassettes were confirmed by PCR amplification and Sanger sequencing. The resulting B. subtilis prsA gene integration strains are summarized in Table 3.

TABLE 3

| B. subtilis- | prsA | Species | |
|---|---|---|---|
| strain | gene | (prsA gene) | Genotype |
| Bs#112 | SEQ ID | B. pumilus | PY79 KO-7S |
| | NO: 2 | | DaprE::PsecA_prsA_Bpu |
| Bs#113 | SEQ ID | B. licheniformis | PY79 KO-7S |
| | NO: 13 | | DaprE::PsecA_prsA_Bli |
| Bs#114 | SEQ ID | B. lentus | PY79 KO-7S |
| | NO: 15 | | DaprE::PsecA_prsA_Ble |
| Bs#115 | SEQ ID | G. stearother- | PY79 KO-7S |
| | NO: 17 | mophilus | DaprE::PsecA_prsA_Gst |

‘D’ denotes deleted

B. subtilis Amylase Expression Strains

Bacillus subtilis strains as listed in Table 3 were made competent as described above for B. subtilis Bs #056. Amylase expression plasmids pAMY029, pAMY031 and pAMY035 were isolated from B. subtilis Bs #056 strain and transformed into the B. subtilis strains PY79 KO-S (control strain) and Bs #112-Bs #115 and plated on LB-agar plates with 20 μg/μl kanamycin. Individual clones were analyzed for correctness of the plasmid DNA by restriction digest and functional enzyme expression was assessed by transfer of individual clones on LB-plates with 2% soluble starch for clearing zone formation of amylase producing strains. The resulting B. subtilis expression strains are listed in Table 4.

TABLE 4
Overview of B. subtilis amylase expression strains

| B. subtilis | Amylase | | Species of |
|---|---|---|---|
| expression | expression | B. subtilis | additional |
| strain | plasmid | strain | prsA gene |
| BES#190 | pAMY029 | PY79 KO-7S | n.a |
| BES#191 | pAMY029 | Bs#112 | B. pumilus |
| BES#192 | pAMY029 | Bs#113 | B. licheniformis |
| BES#193 | pAMY029 | Bs#114 | B. lentus |
| BES#194 | pAMY029 | Bs#115 | G. stearother- |
| | | | mophilus |
| BES#195 | pAMY031 | PY79 KO-7S | n.a |
| BES#196 | pAMY031 | Bs#112 | B. pumilus |
| BES#197 | pAMY031 | Bs#113 | B. licheniformis |
| BES#198 | pAMY031 | Bs#114 | B. lentus |
| BES#199 | pAMY031 | Bs#115 | G. stearother- |
| | | | mophilus |
| BES#200 | pAMY035 | PY79 KO-7S | n.a |
| BES#201 | pAMY035 | Bs#112 | B. pumilus |
| BES#202 | pAMY035 | Bs#113 | B. licheniformis |
| BES#203 | pAMY035 | Bs#114 | B. lentus |
| BES#204 | pAMY035 | Bs#115 | G. stearother- |
| | | | mophilus |

B. licheniformis Strains

Bacillus licheniformis strain Bli #008 (WO2022018260) comprising deletions in the Subtilisin aprE gene, amylase amyB gene, sporulation factor sigF gene (spoIIAC) and poly-gamma glutamate synthesis genes was used to integrate the prsA gene expression cassettes into the genome of B. licheniformis and thereby replacing the chloramphenicol resistance gene of Bacillus licheniformis (SEQ ID NO:7).

For gene deletion/integration in Bacillus licheniformis strains gene deletion/integration plasmids were transformed into E. coli strain Ec #098 made competent according to the method of Chung (Chung, C. T., Niemela, S. L., and Miller, R. H. (1989). One-step preparation of competent Escherichia coli: transformation and storage of bacterial cells in the same solution. Proc. Natl. Acad. Sci. U.S.A. 86, 2172-2175), following selection on LB-agar plates containing 100 μg/ml ampicillin and 30 μg/ml chloramphenicol at 37° C. Plasmid DNA was isolated from individual clones and used for subsequent transfer into Bacillus licheniformis strains. The isolated plasmid DNA carries the DNA methylation pattern of Bacillus licheniformis strains respectively and is protected from degradation upon transfer into Bacillus licheniformis.

B. licheniformis Strains with Integrated Expression Cassettes of the prsA Gene of Various Species.

Electrocompetent Bacillus licheniformis cells were prepared as described above and transformed with 1 μg of pInt20_prsA_Bpu integration plasmid isolated from E. coli Ec #098 following plating on LB-agar plates containing 5 μg/ml erythromycin at 30° C.

The gene integration procedure was performed as described in the following: Plasmid carrying Bacillus licheniformis cells were grown on LB-agar plates with 5 μg/ml erythromycin at 45° C. driving integration of the deletion plasmid via Campbell recombination into the chromosome with one of the homology regions of p pInt20_prsA_Bpu homologous to the sequences 5′ or 3′ of the chloramphenicol cat gene. Clones were picked and cultivated in LB-media without selection pressure at 45° C. for 6 hours, following plating on LB-agar plates with 5 μg/ml erythromycin and incubation overnight at 30° C. Individual clones were picked and screened by colony-PCR analysis with oligonucleotides SEQ ID NO:10 and SEQ ID NO:11 for successful genomic integration of the prsA expression construct at the cat gene locus. Putative integration positive individual clones were picked and taken through two consecutive overnight incubations in LB media without antibiotics at 45° C. to cure the plasmid and plated on LB-agar plates for overnight incubation at 37° C. Single clones were analyzed by colony PCR for successful genomic integration of the prsA expression cassette at the cat gene locus. A single erythromycin-sensitive clone with the correct integrated prsA-B. pumilus expression cassette was isolated and designated Bacillus licheniformis Bli #208.

The construction of other prsA integration strains were conducted as described for Bacillus licheniformis Bli #208. Table 5 summarizes the B. licheniformis strains with integrated prsA expression cassettes.

TABLE 5
B. licheniformis strains with integrated prsA expression cassettes

| B. licheniformis | prsA | Species additional | | Integration |
|---|---|---|---|---|
| strain | gene | prsA gene | Genotype | plasmid |
| Bli#208 | SEQ ID | B. pumilus | DaprE DamyB DsigF Dpga | plnt20_prsA_Bpu |
| | NO: 2 | | DamyB::PaprE_prsA_Bpu | |
| Bli#209 | SEQ ID | B. licheniformis | DaprE DamyB DsigF Dpga | plnt21_prsA_Bli |
| | NO: 13 | | DamyB::PaprE_prsA_Bli | |
| Bli#210 | SEQ ID | B. lentus | DaprE DamyB DsigF Dpga | plnt22_prsA_Ble |
| | NO: 15 | | DamyB::PaprE_prsA_Ble | |
| Bli#211 | SEQ ID | G. stearother- | DaprE DamyB DsigF Dpga | plnt23_prsA_Gst |
| | NO: 17 | mophilus | DamyB::PaprE_prsA_Gst | |

‘D’ denotes deleted

B. licheniformis Amylase Expression Strains

Bacillus licheniformis strains as listed in Table 5 were made competent as described above. Amylase expression plasmids pAMY029, pAMY031, pAMY033 and pAMY035 were isolated from B. subtilis Bs #056 to carry the B. licheniformis specific DNA methylation pattern. Plasmids were transformed in the B. licheniformis Bli #008 control strain (with only the native prsA gene) and the B. licheniformis strains with additionally integrated expression cassettes of prsA genes of B. pumilus, B. licheniformis, B. lentus and G. stearothermophilus respectively. Subsequently the transformed strains were plated on LB-agar plates with 20 μg/μl kanamycin. Individual clones were analyzed for correctness of the plasmid DNA by restriction digest and functional enzyme expression was assessed by transfer of individual clones on LB-plates with 2% soluble starch for clearing zone formation of amylase producing strains. The resulting B. licheniformis expression strains are listed in Table 6.

TABLE 6
B. licheniformis amylase expression strains
with a prsA gene of different species

| B. licheni- | Amylase | B. licheni- | Species of |
|---|---|---|---|
| formis | expression | formis | additional |
| expression strain | plasmid | strain | prsA gene |
| BES#205 | pAMY029 | Bli#008 | n.a. |
| BES#206 | pAMY029 | Bli#208 | B. pumilus |
| BES#207 | pAMY029 | Bli#209 | B. licheniformis |
| BES#208 | pAMY029 | Bli#210 | B. lentus |
| BES#209 | pAMY029 | Bli#211 | G. stearothermophilus |
| BES#210 | pAMY031 | Bli#008 | n.a. |
| BES#211 | pAMY031 | Bli#208 | B. pumilus |
| BES#212 | pAMY031 | Bli#209 | B. licheniformis |
| BES#213 | pAMY031 | Bli#210 | B. lentus |
| BES#214 | pAMY031 | Bli#211 | G. stearothermophilus |
| BES#215 | pAMY033 | Bli#008 | n.a. |
| BES#216 | pAMY033 | Bli#208 | B. pumilus |
| BES#217 | pAMY033 | Bli#209 | B. licheniformis |
| BES#218 | pAMY033 | Bli#210 | B. lentus |
| BES#219 | pAMY033 | Bli#211 | G. stearothermophilus |
| BES#220 | pAMY035 | Bli#008 | n.a. |
| BES#221 | pAMY035 | Bli#208 | B. pumilus |
| BES#222 | pAMY035 | Bli#209 | B. licheniformis |
| BES#223 | pAMY035 | Bli#210 | B. lentus |
| BES#224 | pAMY035 | Bli#211 | n.a. |

In a next step further B. lichenformis amylase expression strains were constructed as just described. Amylase expression plasmids as indicated in the Table 7 below were transformed into the B. licheniformis Bli #008 control strain and the B. licheniformis Bli #208 strain with an additional expression cassette of prsA of B. pumilus integrated in the chromosome.

TABLE 7
B. licheniformis amylase expression strains with
and without the prsA gene of B. pumilus.

| B. licheni- | Amylase | B. licheni- | Species of |
|---|---|---|---|
| formis | expression | formis | additional |
| expression strain | plasmid | strain | prsA gene |
| BES#225 | pAMY038 | Bli#008 | n.a. |
| BES#226 | pAMY038 | Bli#208 | B. pumilus |
| BES#227 | pAMY040 | Bli#008 | n.a. |
| BES#228 | pAMY040 | Bli#208 | B. pumilus |
| BES#229 | pAMY042 | Bli#008 | n.a. |
| BES#230 | pAMY042 | Bli#208 | B. pumilus |
| BES#231 | pAMY045 | Bli#008 | n.a. |
| BES#232 | pAMY045 | Bli#208 | B. pumilus |
| BES#233 | pAMY048 | Bli#008 | n.a. |
| BES#234 | pAMY048 | Bli#208 | B. pumilus |
| BES#235 | pAMY050 | Bli#008 | n.a. |
| BES#236 | pAMY050 | Bli#208 | B. pumilus |
| BES#237 | pAMY053 | Bli#008 | n.a. |
| BES#238 | pAMY053 | Bli#208 | B. pumilus |
| BES#239 | pAMY057 | Bli#008 | n.a. |
| BES#240 | pAMY057 | Bli#208 | B. pumilus |
| BES#241 | pAMY059 | Bli#008 | n.a. |
| BES#242 | pAMY059 | Bli#208 | B. pumilus |
| BES#243 | pAMY061 | Bli#008 | n.a. |
| BES#244 | pAMY061 | Bli#208 | B. pumilus |

### Example 1: Cultivation of Bacillus subtilis Amylase Expression Strains with Coexpression of a prsA Gene of Various Species

Bacillus subtilis amylase expression strains without additional prsA gene (control strain) and with additional psrA genes as listed in Table 4 were cultivated in a microtiter plate-based fed-batch process (Habicher et al., 2019 Biotechnol J.; 15(2)).

All cultivations were conducted in an orbital shaker with a diameter of 25 mm (Innova 42, New Brunswick Scientific, Eppendorf AG; Hamburg, Germany) at 400 rpm. Strains were cultivated in two subsequent precultures in FlowerPlates (MTP-48-OFF, m2p-labs GmbH) for synchronization of growth. The first preculture was carried out in 800 μl TB medium inoculated with a fresh single colony from the strain streaked onto LB agar plates. After 20 h at 30° C., the second preculture containing 800 μl V3 minimal medium (Meissner et al., 2015, Journal of industrial microbiology & biotechnology 42 (9): 1203-1215) was inoculated with 8 μl of the first preculture and cultivated for 24 h at 30° C. Microtiter plate-based fed-batch main cultivations were conducted using 48-well round- and deep-well-microtiter plates with glucose-containing polymer on the bottom of each well (FeedPlate, article number: SMFP08004, Kuhner Shaker GmbH; Herzogenrath, Germany). 70 μl of the second preculture were used to inoculate 700 μl V3-FP minimal medium without glucose supplemented with 5 mM CaCl₂. Main cultures were incubated for 72 h at 35° C. Precultures were covered with a sterile gas-permeable sealing foil (AeraSeal film, Sigma-Aldrich) to avoid contamination. FeedPlates were sealed with a sterile gas-permeable, evaporation reducing foil (F-GPR48-10, m2p-labs GmbH) to reduce evaporation and to avoid contamination.

At the end of the fermentation process, cultivation samples were withdrawn and supernatants prepared by centrifugation and sterile filtration with a 0.2 μm filter. For amylases with poor solubility suitable dilution steps is required prior filtration or sterile filtration. The amylase activity was determined by a method employing the substrate Ethyliden-4-nitrophenyl-α-D-maltoheptaosid (EPS). D-maltoheptaoside is a blocked oligosaccharide which can be cleaved by an endo-amylase. Following the cleavage an alpha-glucosidase liberates a PNP molecule which has a yellow color and thus can be measured by visible spectrophotometry at 405 nm. Kits containing EPS substrate and alpha-glucosidase is manufactured by Roche Costum Biotech (cat. No. 10880078103) and are described in Lorentz K. et al. (2000), Clin. Chem., 46/5: 644-649. The slope of the time dependent absorption-curve is directly proportional to the specific activity (activity per mg enzyme) of the alpha-amylase in question under the given set of conditions.

Each strain was cultivated with six replicates and the values of the enzymatic activities were calculated as the mean of the six replicates for each strain. The mean enzymatic activities of the B. subtilis amylase expression strains with additional prsA gene were normalized to the mean activity values of the corresponding B. subtilis amylase production ‘control strain’ which was set to 100%.

TABLE 8
Amylase of pAMY029 expression plasmid

| B. subtilis | | Relative Amylase | |
|---|---|---|---|
| expression strain | Species prsA gene | activity | CV |
| BES#190 | n.a. | 100% | 21% |
| BES#191 | B. pumilus | 77% | 15% |
| BES#192 | B. licheniformis | 136% | 13% |
| BES#193 | B. lentus | 307% | 17% |
| BES#194 | G. stearothermophilus | 220% | 20% |

TABLE 9
Amylase of pAMY031 expression plasmid

| B. subtilis | | Relative | |
|---|---|---|---|
| expression strain | Species prsA gene | Amylase activity | CV |
| BES#195 | n.a. | 100% | 11% |
| BES#196 | B. pumilus | 128% | 14% |
| BES#197 | B. licheniformis | 121% | 7% |
| BES#198 | B. lentus | 80% | 8% |
| BES#199 | G. stearothermophilus | 133% | 11% |

TABLE 10
Amylase of pAMY035 expression plasmid

| B. subtilis | | Relative Amylase | |
|---|---|---|---|
| expression strain | Species prsA gene | activity | CV |
| BES#200 | n.a. | 100% | 7% |
| BES#201 | B. pumilus | 182% | 16% |
| BES#202 | B. licheniformis | 168% | 15% |
| BES#203 | B. lentus | 182% | 20% |
| BES#204 | G. stearothermophilus | 171% | 28% |

The productivity of the Amylase of pAMY029 increased by 30% to 200% in the presence of additional expression of prsA genes of B. licheniformis, B. lentus and G. stearothermophilus respectively with the PrsA of B. lentus having the greatest increase on Amylase productivity (Table 8). The additional expression of the prsA gene of B. pumilus has a negative impact on the productivity of the Amylase of pAMY029 in B. subtilis.

The Amylase productivity of the Amylase of pAMY031 increased by 20%-30% in presence of the additional expression of prsA genes of B. licheniformis, B. pumilus and G. stearothermophilus whereas in contrast to the Amylase of pAMY029 the additional expression of the prsA gene of B. lentus had a negative impact on the productivity of the Amylase of pAMY031 in B. subtilis (Table 9).

The Amylase productivity of the B. licheniformis Amylase AmyL of pAMY035 in B. subtilis increased by 70%-80% in presence of the additional expression of the prsA genes from all tested species, namely B. licheniformis, B. pumilus, B. lentus and G. stearothermophilus (Table 10).

### Example 2: Cultivation of Bacillus licheniformis Amylase Expression Strains with Coexpression of a prsA Gene of Various Species

Bacillus licheniformis amylase expression strains without additional prsA gene (control strain) and with additional prsA genes as listed in Table 6 were cultivated under conditions in a microtiter plate-based fed-batch process as described for B. subtilis (Example 1).

At the end of the fermentation process, cultivation samples were withdrawn and supernatants prepared by centrifugation and sterile filtration with a 0.2 μm filter. The amylase activity was determined as described in Example 1. The enzymatic activities were calculated as the mean of the six replicates for each strain. The mean enzymatic activities of the B. licheniformis amylase expression strains with additional prsA gene were normalized to the mean activity values of the corresponding B. licheniformis amylase expression ‘control strain’ which was set to 100%.

TABLE 11
Amylase of pAMY029 expression plasmid

| B. licheniformis | | Relative Amylase | |
|---|---|---|---|
| expression strain | Species prsA gene | activity | CV |
| BES#205 | n.a. | 100% | 16% |
| BES#206 | B. pumilus | 416% | 16% |
| BES#207 | B. licheniformis | 220% | 14% |
| BES#208 | B. lentus | 250% | 9% |
| BES#209 | G. stearothermophilus | 144% | 9% |

TABLE 12
Amylase of pAMY031 expression plasmid

| B. licheniformis | | Relative Amylase | |
|---|---|---|---|
| expression strain | Species prsA gene | activity | CV |
| BES#210 | n.a. | 100% | 7% |
| BES#211 | B. pumilus | 333% | 5% |
| BES#212 | B. licheniformis | 256% | 3% |
| BES#213 | B. lentus | 126% | 4% |
| BES#214 | G. stearothermophilus | 145% | 6% |

TABLE 13
Amylase of pAMY033 expression plasmid

| B. licheniformis | | Relative Amylase | |
|---|---|---|---|
| expression strain | Species prsA gene | activity | CV |
| BES#215 | n.a. | 100% | 8% |
| BES#216 | B. pumilus | 262% | 5% |
| BES#217 | B. licheniformis | 196% | 7% |
| BES#218 | B. lentus | 138% | 10% |
| BES#219 | G. stearothermophilus | 146% | 9% |

TABLE 14
Amylase of pAMY035 expression plasmid

| B. licheniformis | | Relative Amylase | |
|---|---|---|---|
| expression strain | Species prsA gene | activity | CV |
| BES#220 | n.a. | 100% | 12% |
| BES#221 | B. pumilus | 126% | 8% |
| BES#222 | B. licheniformis | 166% | 11% |
| BES#223 | B. lentus | 159% | 7% |
| BES#224 | G. stearothermophilus | 135% | 10% |

The productivity of the Amylase of pAMY029 in B. licheniformis increased by 40% to over 316% in the presence of the additional expression of prsA genes of B. pumilus, B. licheniformis, B. lentus and G. stearothermophilus respectively (Table 11). In contrast to the results with B. subtilis where the expression of the prsA gene of B. pumilus (BES #191) had a negative impact on the productivity of the Amylase of pAMY029, the expression of the prsA gene of B. pumilus in B. licheniformis surprisingly showed the highest increase of productivity of the Amylase of pAMY029.

Similar to the results of the Amylase of pAMY029, the Amylase productivities in B. lichenformis of the Amylase of pAMY031 (Table 12) and Amylase of pAMY033 (Table 13) increased highest with the expression of the prsA gene of B. pumilus in comparison to the expression of the prsA gene of the other species.

The Amylase productivity of the B. licheniformis Amylase AmyL of pAMY035 in B. licheniformis increased by 26%-66% Table 14) in the presence of the additional expression of the prsA genes from all tested species, namely B. licheniformis, B. pumilus, S. lentus and G. stearothermophilus.

### Example 3: Cultivation of Bacillus licheniformis Amylase Expression Strains with Coexpression of the prsA Gene of B. pumilus

To further analyze the surprisingly strong impact of the additional expression of the prsA gene of S. pumilus on heterologous amylase production in B. licheniformis, the Amylase productivity was analyzed for various different amylases in either B. licheniformis Bli #008 control strain (only native prsA gene) or in B. licheniformis Bli #208 strain with an additional expression cassette of the prsA gene of S. pumilus.

MTP-based fed-batch cultivations were conducted as described in Example 2 and the enzymatic activities determined.

Table 15 shows the mean enzymatic activity values of six replicates each of the B. licheniformis amylase expression strains (Table 7) with the additional prsA gene of B. pumilus which were normalized to the mean activity values of the corresponding B. licheniformis amylase expression ‘control strain’ which was set to 100%.

TABLE 15
Increased Amylase production in B. licheniformis
with additional prsA of B. pumilus

| Amylase | native prsA | | additional prsA B. pumilus | |
|---|---|---|---|---|
| expression | Relative Amylase | | Relative Amylase | |
| plasmid | activity | CV | activity | CV |
| pAMY031 | 100% | 14% | 314% | 20% |
| pAMY038 | 100% | 23% | 189% | 10% |
| pAMY040 | 100% | 13% | 172% | 18% |
| pAMY042 | 100% | 26% | 179% | 23% |
| pAMY045 | 100% | 17% | 94% | 14% |
| pAMY048 | 100% | 37% | 360% | 13% |
| pAMY050 | 100% | 12% | 296% | 10% |
| pAMY053 | 100% | 18% | 187% | 21% |
| pAMY057 | 100% | 31% | 99% | 7% |
| pAMY059 | 100% | 17% | 176% | 9% |
| pAMY061 | 100% | 17% | 189% | 39% |

The additional expression of the prsA gene of B. pumilus in B. licheniformis surprisingly greatly increased the Amylase productivity for various Bacillus derived Amylases of amylase expression plasmids pAMY031, pAMY038, pAMY040, pAMY042, pAMY048, pAMY050, pAMY053, pAMY059 and pAMY061.

1. A Bacillus licheniformis host cell expressing
a) a first polypeptide having peptidyl-prolyl cis-trans isomerase (EC 5.2.1.8) activity, said first polypeptide comprising an amino acid sequence being a least 81% identical to SEQ ID NO: 1, and
b) a second polypeptide having amylase activity, wherein said second polypeptide is heterologous to said Bacillus licheniformis host cell.

2. The Bacillus licheniformis host cell of claim 1, wherein said first polypeptide comprises an amino acid sequence being at least 90% identical to SEQ ID NO: 1.

3. The Bacillus licheniformis host cell of claim 1, wherein said second polypeptide has alpha amylase activity (EC 3.2.1.1) or maltogenic alpha-amylase activity (EC 3.2.1.133).

4. The Bacillus licheniformis host cell of claim 1, wherein said second polypeptide comprises an amino acid sequence being at least 86%, at least 87%, at least 88%, at least 89%, at least 90%, at least 91%, at least 92%, at least 93%, at least 94%, at least 95%, at least 96%, at least 97%, at least 98%, at least 99% or at least 99.5% or 100% identical to SEQ ID NO: 29, 35, 38, 40, 42, 48, 50, 53, 59 or 61.

5. The Bacillus licheniformis host cell of claim 1, wherein said second polypeptide comprises an amino acid sequence being at least 95% at least 96%, at least 97%, at least 98%, at least 99%, at least 99.5%, or 100% identical to SEQ ID NO: 29, 35, 38, 40, 42, 48, 50, 53, 59 or 61.

6. The Bacillus licheniformis host cell of claim 1, wherein the host cell comprises:
a) a first expression cassette for said first polypeptide, said first expression cassette comprising a promoter, operably linked to a first polynucleotide encoding said first polypeptide, and optionally a terminator, and
b) a second expression cassette for said second polypeptide, said second expression cassette comprising a promoter, operably linked to a second polynucleotide encoding said second polypeptide, and optionally a terminator.

7. The Bacillus licheniformis host cell of claim 6, wherein the promoter of the first expression cassette and/or the promoter of the second expression cassette is an inducer independent promoter or an inducible promoter.

8. The Bacillus licheniformis host cell of claim 6, wherein the first expression cassette and/or second expression cassette is/are present in the host cell on a plasmid or is/are stably integrated into the chromosomal DNA of the host cell.

9. The Bacillus licheniformis host cell of claim 1, wherein said second polypeptide is secreted.

10. The Bacillus licheniformis host cell of claim 1, wherein said second polypeptide comprises a signal peptide.

11. The Bacillus licheniformis host cell of claim 1, 1, wherein the host cell is a host cell from a strain selected from the group consisting of Bacillus licheniformis strains ATCC 14580, ATCC 31972, ATCC 53757, ATCC 53926, ATCC 55768, DSM 13, DSM 394, DSM 641, DSM 1913, DSM 11259, and DSM 26543.

12. A method of producing a polypeptide having amylase activity, comprising
a) providing the Bacillus licheniformis host cell as defined in claim 1, and
b) cultivating the host cell under conditions that allow for expressing said polypeptide having amylase activity, and optionally,
c) obtaining or purifying said polypeptide having amylase activity.

13. A method of producing the Bacillus licheniformis host cell of claim 1, comprising
a) providing a Bacillus licheniformis host cell, and
b) introducing into the host cell provided in step a) a first polynucleotide encoding the first polypeptide as defined in claim 1 and b) a second polynucleotide encoding the second polypeptide as defined in claim 1.

14. The method of claim 13, wherein in step b) comprises introducing a first expression cassette comprising a promoter, operably linked to the first polynucleotide and optionally a terminator, and a second expression cassette comprising a promoter, operably linked to the second polynucleotide, and optionally a terminator, into the host cell.

15. A method for increasing the production of a second polypeptide having alpha amylase activity as defined in claim 1 in a Bacillus licheniformis host cell, comprising co-expressing a first polypeptide having peptidyl-prolyl cis-trans isomerase (EC 5.2.1.8) activity as defined in claim 1, and/or of a polynucleotide encoding said first polypeptide.
